(19) United States
(12) Patent Application Publication
(10) Pub. No.: US 2023/0077584 A1
(43) Pub. Date: Mar. 16, 2023

(54) METHODS AND COMPOSITIONS FOR MODULATING ENTEROENDOCRINE CELLS

(71) Applicant: Flagship Pioneering Innovations VI, LLC, Cambridge, MA (US)

(72) Inventors: Morag Helen STEWART, Boston, MA (US); Angeliki CHALKIADAKI, Brookline, MA (US); Fabian Alexander WOLF, Munich (DE); Sophie Andrea TRITSCHLER, Munich (DE); Nicholas McCartney PLUGIS, Duxbury, MA (US)

(21) Appl. No.: 17/941,632

(22) Filed: Sep. 9, 2022

Related U.S. Application Data
(60) Provisional application No. 63/242,226, filed on Sep. 9, 2021.

(51) Int. Cl. C12N 5/071; A61P 3/04

(52) U.S. Cl. CPC C12N 5/068; A61P 3/04; C12N 2501/00

(57) ABSTRACT

The present disclosure relates, inter alia, to perturbagens and methods for directing a change in the cell state of an intestinal stem cell. It also relates to methods for increasing a quantity of enteroendocrine cells, goblet progenitors, goblet cells, and/or Paneth cells or immediate progenitors thereof and/or the ratios thereof. Further, the present disclosure relates to methods for treating diseases or disorders characterized by, at least, abnormal function, abnormal ratios and/or abnormal numbers of enteroendocrine cells, goblet progenitors, goblet cells, and/or Paneth cells, or immediate progenitors thereof.

### CROSS-REFERENCE TO RELATED APPLICATIONS

This application claims priority to and benefit of U.S. Provisional Patent Application No. 63/242,226 filed on Sep. 9, 2021, the contents of which is hereby incorporated by reference in its entirety.

### BACKGROUND

An understanding of cellular mechanisms relating to development of enteroendocrine cells and their progenitors, as well as methods and agents for directing changes in development of these cells, may be useful for treating diseases or disorders, including those underscored by abnormal amounts or ratio of various cell types in the enteroendocrine cell lineage. Currently, there is an unmet need for such methods and agents that can be used for the treatment of such diseases and disorders.

### SUMMARY

Accordingly, in various aspects, the present disclosure provides methods for directing a change in the cell state of a progenitor cell (including, e.g., an intestinal stem cell) and agents that are suitable for achieving such change. These agents are described herein as perturbagens. The present disclosure also provides methods for increasing the quantity, function, and/or the ratios of enteroendocrine cells, goblet progenitors, Paneth cells, and/or goblet cells. The present disclosure further provides methods for treating diseases or disorders characterized by, at least, abnormal function (e.g., abnormal amount of mucous production and secretion), abnormal ratios, and/or abnormal numbers of enteroendocrine cells, goblet progenitors, Paneth cells, and/or goblet cells, or immediate progenitors thereof (e.g., intestinal stem cells or enteroendocrine cell progenitors). In various aspects, the cellular manipulations described herein are guided and/or mediated by gene signatures that reflect a cellular state and/or capacity for transitioning of a cell from one state to a different cellular state.

In some aspects, the present disclosure is related to a method for directing a change in cell state of a progenitor cell comprising: contacting a population of cells comprising a progenitor cell with at least one perturbagen selected from Table 2, or a variant thereof; wherein the at least one perturbagen is capable of altering a gene signature in the progenitor cell; and wherein the progenitor cell is an intestinal stem cell.

In some aspects, the present disclosure is related to a method for directing a change in cell state of a progenitor cell, comprising: contacting a population of cells comprising a progenitor cell with at least one perturbagen capable of altering a gene signature in the progenitor cell; wherein altering the gene signature comprises an increase in expression and/or activity in the progenitor cell of one or more genes designated as an “up” gene in the gene directionality column of Table 1 and/or a decrease in expression and/or activity in the progenitor cell of one or more genes designated as a “down” gene in the gene directionality column of Table 1; and wherein the progenitor cell is an intestinal stem cell.

In some aspects, the present disclosure is related to a method for directing a change in cell state of a progenitor cell, comprising: contacting a population of cells comprising a progenitor cell with at least one perturbagen selected from Table 2, or a variant thereof, and capable of altering a gene signature in the progenitor cell; wherein altering the gene signature comprises an increase in expression and/or activity in the progenitor cell of one or more genes designated as an “up” gene in the gene directionality column of Table 1 and/or a decrease in expression and/or activity in the progenitor cell of one or more genes designated as a “down” gene in the gene directionality column of Table 1; and wherein the progenitor cell is an intestinal stem cell.

In embodiments, altering the gene signature comprises a change in expression and/or activity of one or more genes in the progenitor cell of a network module designated in the network module column of Table 1.

In embodiments, the change in cell state provides an increase in the number of one or more of enteroendocrine cells, goblet progenitors, goblet cells, and Paneth cells.

In embodiments, the change in cell state provides an increase in the number of enteroendocrine cells.

In embodiments, the increase in the number of enteroendocrine cells is relative to the number of enteroendocrine cells obtained from a population of progenitor cells that is not contacted with the at least one perturbagen and/or relative to the number of enteroendocrine cells obtained from a population of progenitor cells prior to contacting with the at least one perturbagen.

In embodiments, the number of progenitor cells is decreased.

In embodiments, the decrease in the number of progenitor cells is relative to the number of progenitor cells in a population of progenitor cells that is not contacted with the at least one perturbagen and/or relative to the number of progenitor cells in the population prior to contacting with the at least one perturbagen.

In embodiments, the number of progenitor cells is increased.

In embodiments, the increase in the number of progenitor cells is relative to the number of progenitor cells in a population of progenitor cells that is not contacted with the at least one perturbagen and/or relative to the number of progenitor cells in the population prior to contacting with the at least one perturbagen.

In embodiments, the number of goblet progenitors, goblet cells, Paneth cells, and/or enteroendocrine cells is increased after contacting the population of cells comprising a progenitor cell with the at least one perturbagen.

In embodiments, the number of goblet progenitors, goblet cells, Paneth cells, enterocyte progenitor cells, and/or enterocytes is decreased.

In embodiments, the number of goblet progenitors, goblet cells, Paneth cells, and/or enteroendocrine cells is increased.

In embodiments, the at least one perturbagen selected from Table 2, or a variant thereof, comprises at least 2, at least 3, at least 4, at least 5, at least 6, at least 7, at least 8, at least 9, at least 10, at least 11, or all 12 perturbagens selected from Table 2, or variants thereof.

In embodiments, the one or more genes are selected from the genes designated as a “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, or 68 genes selected from the genes designated as a “down” gene in the gene directionality column of Table 1.

In embodiments, the one or more genes selected from Table 1 comprises at least one of CD44, DCTD, CDK6, GAA, DDB2, HMGA2, ST7, SLC35F2, MLEC, DPH2, MBNL1, JADE2, MIF, SLC5A6, C2CD2, CRTAP, ATF1, PPIE, ADCK3, HES1, ATP1B1, TIMM9, MYC, MAP3K4, CHERP, TBP, DAG1, TEX10, BAG3, NET1, FZD7, RAD9A, NUDT9, PIK3R4, MRPL12, FPGS, ANXA7, HN1L, METRN, LYN, TGFBR2, STAT5B, RAC2, MALT1, DHX29, EPHB2, CDC25B, PIH1D1, GTPBP8, RBM15B, ELOVL6, IKBKAP, SLC25A13, HSPD1, TSEN2, HEATR1, ME2, BACE2, RFX5, BDH1, PPARG, SLC37A4, NNT, DNM1, ICMT, ETFB, NCK2, and CCND1.

In embodiments, contacting the population of progenitor cells occurs in vitro or ex vivo or in vivo in a subject.

In some aspects, the present disclosure is related to a perturbagen for use in a method of the present disclosure.

In some aspects, the present disclosure is related to a pharmaceutical composition comprising a perturbagen of the present disclosure.

In some aspects, the present disclosure is related to a method for promoting the formation of an enteroendocrine cell, or an immediate progenitor thereof, comprising: exposing a starting population of intestinal stem cells to a perturbation having a perturbation signature that promotes the transition of the starting population of intestinal stem cells into a goblet progenitor cell or an enteroendocrine cell, wherein the perturbation signature comprises increased expression and/or activity in the intestinal stem cell of one or more of genes designated as an “up” gene in the gene directionality column of Table 1 and/or a decreased expression and/or activity in the intestinal stem cell of one or more genes designated as a “down” gene in the gene directionality column of Table 1.

In embodiments, the perturbation signature comprises an increase in expression and/or activity of one or more genes in the progenitor cell of an activation of a network module designated in the network module column of Table 1.

In some aspects, the present disclosure is related to a method for treating a disease or disorder characterized by an abnormal number of enteroendocrine cells, comprising: (a) administering to a patient in need thereof a therapeutically effective amount of at least one perturbagen selected from Table 2, or a variant thereof, wherein the at least one perturbagen is capable of changing a gene signature in a progenitor cell, or (b) administering to a patient in need thereof a cell, the cell having been contacted with at least one perturbagen selected from Table 2, or a variant thereof, wherein the at least one perturbagen is capable of changing a gene signature in a progenitor cell.

In embodiments, the disease or disorder is caused by an enteroendocrine cell deficiency.

In embodiments, the disease or disorder is selected from Type II Diabetes, obesity, weight loss, intestinal inflammation (e.g. inflammatory bowel disease, infection, colorectal cancer or food allergies), nonalcoholic fatty liver disease, and cardiovascular complications of diabetes.

In some aspects, the present disclosure is related to a method for treating a disease or disorder characterized by an abnormal ratio of enteroendocrine cells to intestinal stem cells, comprising: (a) administering to a patient in need thereof at least one perturbagen selected from Table 2, or a variant thereof, wherein the at least one perturbagen is capable of changing a gene signature in an intestinal stem cell or (b) administering to a patient in need thereof a cell, the cell having been contacted with at least one perturbagen selected from Table 2, or a variant thereof, wherein the at least one perturbagen is capable of changing a gene signature in an intestinal stem cell.

In embodiments, the abnormal ratio comprises a decreased number of enteroendocrine cells and/or an increased number of intestinal stem cells.

In embodiments, the disease or disorder is selected from Type II Diabetes, obesity, weight loss, intestinal inflammation (e.g. inflammatory bowel disease, infection, colorectal cancer or food allergies), nonalcoholic fatty liver disease, and cardiovascular complications of diabetes.

In embodiments, the at least one perturbagen is capable of changing a gene signature in a progenitor cell.

In embodiments, the patient is selected by steps comprising: obtaining from the patient having the disease or disorder a sample of cells comprising at least one intestinal stem cell; and contacting the sample of cells with least one perturbagen selected from Table 2, or a variant thereof, wherein the at least one perturbagen alters a gene signature in the sample of cells.

In embodiments, the patient is selected by steps comprising: obtaining from a subject having the disease or disorder a sample of cells comprising at least one intestinal stem cell; and contacting the sample of cells with at least one perturbagen capable of altering a gene signature in an intestinal stem cell, wherein the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1.

In embodiments, the patient is selected by steps comprising: obtaining from a subject having the disease or disorder a sample of cells comprising an intestinal stem cell; and contacting the sample of cells with at least one perturbagen selected from Table 2, or a variant thereof; wherein the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1.

In embodiments, the perturbagen causes an increase in expression and/or activity of one or more genes in the progenitor cell of an activation of a network module designated in the network module column of Table 1.

In some aspects, the present disclosure is related to a method for selecting a patient of any one of the methods of the present disclosure, comprising: obtaining from a subject having the disease or disorder a sample of cells comprising an intestinal stem cell; and contacting the sample of cells with least one perturbagen selected from Table 2, or a variant thereof, wherein when the at least one perturbagen alters a gene signature in the sample of cells, the subject is selected as a patient.

In some aspects, the present disclosure is related to a method for selecting a patient of any one of the methods of the present disclosure, comprising: obtaining from a subject having the disease or disorder a sample of cells comprising an intestinal stem cell; and contacting the sample of cells with at least one perturbagen capable of altering a gene signature in an intestinal stem cell, wherein when the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1, the subject is selected as a patient.

In some aspects, the present disclosure is related to a method for selecting a patient of any one of the methods of the present disclosure, comprising: obtaining from a subject having the disease or disorder a sample of cells comprising an intestinal stem cell; and contacting the sample of cells with at least one perturbagen selected from Table 2, or a variant thereof; wherein when the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1, the subject is selected as a patient.

In embodiments, the perturbagen causes an increase in expression and/or activity of one or more genes in the progenitor cell of an activation of a network module designated in the network module column of Table 1.

In some aspects, the present disclosure is related to use of a perturbagen of Table 2, or a variant thereof in the manufacture of a medicament for treating a disease or disorder characterized by an abnormal ratio of enteroendocrine cells to intestinal stem cells.

In some aspects, the present disclosure is related to use of a perturbagen of Table 2, or a variant thereof in the manufacture of a medicament for treating a disease or disorder characterized by an abnormal ratio of enteroendocrine cells to enterocytes, Paneth cells and/or goblet cells.

In some aspects, the present disclosure is related to a method of identifying a candidate perturbation for promoting the transition of a starting population of intestinal stem cells into enteroendocrine cells or immediate progenitors thereof, the method comprising: exposing the starting population of intestinal stem cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of intestinal stem cells into enteroendocrine cells or immediate progenitors thereof following exposure of the population of cells to the perturbation; and identifying the perturbation as a candidate perturbation for promoting the transition of a population of intestinal stem cells into enteroendocrine cells or immediate progenitors thereof based on the perturbation signature, wherein the perturbation signature is an increase in expression and/or activity in the intestinal stem cell of one or more genes designated as an “up” gene in the gene directionality column of Table 1, and/or a decrease in expression and/or activity in the intestinal stem cell of one or more genes designated as a “down” gene in the gene directionality column of Table 1.

In embodiments, the perturbation signature is an increase in expression and/or activity in the progenitor cell of an activation of a network module designated in the network module column of Table 1.

In some aspects, the present disclosure is related to a method for making a therapeutic agent for a disease or disorder selected from Type II Diabetes, obesity, weight loss, intestinal inflammation (e.g. inflammatory bowel disease, infection, colorectal cancer or food allergies), nonalcoholic fatty liver disease, and cardiovascular complications of diabetes, comprising: (a) identifying a candidate perturbation according to a method of the disclosure, and (b) formulating the candidate perturbation as a therapeutic agent for the treatment of the disease or disorder.

In some aspects, the present disclosure is related to a method for directing a change in cell state of a progenitor cell, comprising: contacting a population of cells comprising a progenitor cell with at least one perturbagen capable of altering a gene signature in the progenitor cell; wherein altering the gene signature comprises an increase in expression and/or activity in the progenitor cell of one or more genes designated as an “up” gene in the gene directionality column of Table 1 and/or a decrease in expression and/or activity in the progenitor cell of one or more genes designated as a “down” gene in the gene directionality column of Table 1; wherein the progenitor cell is an intestinal stem cell and wherein the change in cell state provides an increase in the number of one or more of enteroendocrine cells, goblet progenitors, goblet cells, and Paneth cells.

In embodiments, the change in cell state provides an increase in the number of enteroendocrine cells.

In embodiments, the increase in the number of enteroendocrine cells is relative to the number of enteroendocrine cells obtained from a population of progenitor cells that is not contacted with the at least one perturbagen and/or relative to the number of enteroendocrine cells obtained from a population of progenitor cells prior to contacting with the at least one perturbagen.

In embodiments, the at least one perturbagen is selected from Table 2, or a variant thereof. In embodiments, the at least one perturbagen selected from Table 2, or a variant thereof, comprises at least 2, at least 3, at least 4, at least 5, at least 6, at least 7, at least 8, at least 9, at least 10, at least 11, or all 12 perturbagens selected from Table 2, or variants thereof.

In embodiments, the one or more genes selected from Table 1 comprises at least one of CD44, DCTD, CDK6, GAA, DDB2, HMGA2, ST7, SLC35F2, MLEC, DPH2, MBNL1, JADE2, MIF, SLC5A6, C2CD2, CRTAP, ATF1, PPIE, ADCK3, HES1, ATP1B1, TIMM9, MYC, MAP3K4, CHERP, TBP, DAG1, TEX10, BAG3, NET1, FZD7, RAD9A, NUDT9, PIK3R4, MRPL12, FPGS, ANXA7, HN1L, METRN, LYN, TGFBR2, STAT5B, RAC2, MALT1, DHX29, EPHB2, CDC25B, PIH1D1, GTPBP8, RBM15B, ELOVL6, IKBKAP, SLC25A13, HSPD1, TSEN2, HEATR1, ME2, BACE2, RFX5, BDH1, PPARG, SLC37A4, NNT, DNM1, ICMT, ETFB, NCK2, and CCND1.

Any aspect or embodiment disclosed herein can be combined with any other aspect or embodiment as disclosed herein.

### BRIEF DESCRIPTION OF THE DRAWINGS

FIG. 1 shows a t-distributed stochastic neighbor embedding (t-SNE) plot illustrating the predictions that drive the transition of cells. Single cell manifold, labelled with cell states, are shown.

FIG. 2 shows mouse intestinal organoids that were treated with the indicated compounds. DAPT alone or combination of DAPT with IWP2 or CHIR99021 induces the expression of chromogranin A and chromogranin B genes, markers of enteroendocrine cells.

FIG. 3 shows data for mice that were treated with indicated small molecules targeting enteroendocrine cells. The data shows an increase in the percentage of enteroendocrine cells in colon. **p<0.005, ***p<0.001.

### DETAILED DESCRIPTION

The present disclosure is based, in part, on the discovery that cells of intestinal lineages comprising goblet progenitors, Paneth cells, enteroendocrine cells, goblets cells, enterocyte progenitors, enterocytes, and their progenitors can be characterized by specific gene signatures. Additionally, the present disclosure is based on the discovery that certain active agents (i.e., perturbagens) can alter these specific gene signatures. Such alteration is associated with the acquisition of specific cell states by cells of enteroendocrine lineages. These perturbagens are, in some instance, useful as therapeutics and derive benefit by directing the intestinal stem cells towards goblet progenitors and/or enteroendocrine cells.

Genes Signatures

Cell state transitions (i.e., a transition in a cell's state from a first cell state to a second cell state, e.g., differentiation) are characterized by a change in expression of genes in the cell. Changes in gene expression may be quantified as, e.g., an increase in mRNA expressed for a specific gene or a decrease in mRNA expressed for another specific gene; especially significant here may be mRNAs that encode transcription factors. Collectively, the sum of multiple differences in gene expression between one cell type or cells of one lineage relative to another cell type or cells of another lineage are referred to herein as a gene signature.

Any one of a number of methods and metrics may be used to identify gene signatures. Non-limiting examples include single cell and bulk RNA sequencing with or without prior cell sorting (e.g., fluorescence activated cell sorting (FACS) and flow cytometry). When developing a gene signature, it may useful to first characterize the cell type or cells of a specific lineage by surface proteins that are characteristic of the cell type or cells of a specific lineage.

Knowing the gene signature for each cell type or cells of a specific lineage provides insight into what genes impact or are associated with the process of transition to other cell types and/or differentiation of progenitor cells.

Gene signatures can be used to identify particular cells as being on-lineage, and other cells as being “progenitor” cells or intermediate cells along a transition trajectory towards the on-lineage cell type.

Genes that are differentially expressed and positively associated with the promotion of enteroendocrine cell lineage progression and/or goblet progenitor cell differentiation are listed in Table 1.

TABLE 1

| | | | Gene_Di- | |
|---|---|---|---|---|
| | Gene | Gene_Entrez_ID | rectionality | Network_Module |
| 0 | NPDC1 | 56654 | up | 3 |
| 1 | CD44 | 960 | down | 3 |
| 2 | DCTD | 1635 | down | 3 |
| 3 | CDK6 | 1021 | down | 3 |
| 4 | GAA | 2548 | down | 3 |
| 5 | DDB2 | 1643 | down | 3 |
| 6 | HMGA2 | 8091 | down | 3 |
| 7 | ST7 | 7982 | down | 3 |
| 8 | SLC35F2 | 54733 | down | 3 |
| 9 | MLEC | 9761 | down | 3 |
| 10 | DPH2 | 1802 | down | 3 |
| 11 | MBNL1 | 4154 | down | 3 |
| 12 | JADE2 | 23338 | down | 3 |
| 13 | MIF | 4282 | down | 3 |
| 14 | SLC5A6 | 8884 | down | 3 |
| 15 | C2CD2 | 25966 | down | 3 |
| 16 | CRTAP | 10491 | down | 3 |
| 17 | ATF1 | 466 | down | 3 |
| 18 | PPIE | 10450 | down | 3 |
| 19 | ADCK3 | 56997 | down | 3 |
| 20 | SOX4 | 6659 | up | 5 |
| 21 | BAMBI | 25805 | up | 5 |
| 22 | HES1 | 3280 | down | 5 |
| 23 | ATP1B1 | 481 | down | 5 |
| 24 | TIMM9 | 26520 | down | 5 |
| 25 | MYC | 4609 | down | 5 |
| 26 | MAP3K4 | 4216 | down | 5 |
| 27 | CHERP | 10523 | down | 5 |
| 28 | TBP | 6908 | down | 5 |
| 29 | DAG1 | 1605 | down | 5 |
| 30 | TEX10 | 54881 | down | 5 |
| 31 | BAG3 | 9531 | down | 5 |
| 32 | NET1 | 9423 | down | 5 |
| 33 | FZD7 | 8324 | down | 5 |
| 34 | RAD9A | 5883 | down | 5 |
| 35 | NUDT9 | 53343 | down | 5 |
| 36 | PIK3R4 | 30849 | down | 5 |
| 37 | DRAP1 | 10589 | up | 6 |
| 38 | SLC25A4 | 291 | up | 6 |
| 39 | MRPL12 | 6182 | down | 6 |
| 40 | FPGS | 2356 | down | 6 |
| 41 | ANXA7 | 310 | down | 6 |
| 42 | HN1L | 90861 | down | 6 |
| 43 | METRN | 79006 | down | 6 |
| 44 | LYN | 4067 | down | 6 |
| 45 | TGFBR2 | 7048 | down | 6 |
| 46 | STAT5B | 6777 | down | 6 |
| 47 | RAC2 | 5880 | down | 6 |
| 48 | MALT1 | 10892 | down | 6 |
| 49 | DHX29 | 54505 | down | 6 |
| 50 | EPHB2 | 2048 | down | 6 |
| 51 | CDC25B | 994 | down | 6 |
| 52 | PIH1D1 | 55011 | down | 6 |
| 53 | CDK4 | 1019 | up | 8 |
| 54 | GTPBP8 | 29083 | down | 8 |
| 55 | RBM15B | 29890 | down | 8 |
| 56 | ELOVL6 | 79071 | down | 8 |
| 57 | IKBKAP | 8518 | down | 8 |
| 58 | SLC25A13 | 10165 | down | 8 |
| 59 | HSPD1 | 3329 | down | 8 |
| 60 | TSEN2 | 80746 | down | 8 |
| 61 | HEATR1 | 55127 | down | 8 |
| 62 | ME2 | 4200 | down | 8 |
| 63 | BACE2 | 25825 | down | 8 |
| 64 | RFX5 | 5993 | down | 8 |
| 65 | BDH1 | 622 | down | 8 |
| 66 | PPARG | 5468 | down | 8 |
| 67 | SLC37A4 | 2542 | down | 8 |
| 68 | SMARCD2 | 6603 | up | 9 |
| 69 | NNT | 23530 | down | 9 |
| 70 | DNM1 | 1759 | down | 9 |
| 71 | ICMT | 23463 | down | 9 |
| 72 | ETFB | 2109 | down | 9 |
| 73 | NCK2 | 8440 | down | 9 |
| 74 | CCND1 | 595 | down | 9 |

The genes listed in Table 1 and classified as “up” in the gene directionality column of Table 1 show an increase in expression in the cell state change. The genes listed in Table 1 and classified as “down” in the gene directionality column of Table 1 show a decrease in expression in the cell state change.

In Table 1 and associated embodiments:
1. “Gene ID”: at the time of filing the present disclosure, the World Wide Web at ncbi.nlm.nih.gov/gene provides a description of and the nucleic acid sequence for each GeneID listed in Table 1; the contents of each of which is incorporated herein by reference in its entirety.
2. “Up” indicates a gene for which an increase in expression and/or activity in the progenitor cell is associated with the gene signature.
3. “Down” indicates a gene for which a decrease in expression and/or activity in the progenitor cell is associated with the gene signature.
4. A “network module” (sometimes also referred to as “module”) is a set of genes whose activity and/or expression are mutually predictive and, individually and collectively, are correlated with regard to a cell state change, which correlation may be positive or negative. That is, a module may contain genes that are positively associated with the cell state transition-such that an increase in expression and/or activity of the gene associated with the cell state transition; as well as genes that are negatively associated with the cell state transition such that a decrease in expression and/or activity of the gene associated with the cell state transition.

In certain embodiments, a network module includes genes in addition (or substituted for) to those exemplified in Table 1, which should be viewed as illustrative and not limiting unless expressly provided, namely with genes with correlated expression. A correlation, e.g., by the method of Pearson or Spearman, is calculated between a query gene expression profile for the desired cell state transition and one or more of the exemplary genes recited in the module. Those genes with a correlation with one or more genes of the module of at significance level below p=0.05 (e.g., 0.04, 0.03, 0.02, 0.01, 0.005, 0.001, 0.0005, 0.0001, or less) can be added to, or substituted for, other genes in the module.

“Activation of a network module” refers to a perturbation that modulates expression and/or activity of 2 or more genes (e.g., 3, 4, 5, 6 . . . genes; or about 10, 20, 25, 30, 40, 50, 60, 70, 75, 80, 85, 90, 95, or 100%) within a module, which modulation may be an increase or decrease in expression and/or activity of the gene as consonant with the modules described in Table 1. In certain embodiments, a perturbation activates multiple network modules for the desired cell state transition, such as 2, 3, 4, or more modules.

In some embodiments, one or more genes of network module 3 are modulated. In some embodiments, the present disclosure relates to the activation of network module 3, e.g., one or more of (inclusive of all of) NPDC1, CD44, DCTD, CDK6, GAA, DDB2, HMGA2, ST7, SLC35F2, MLEC, DPH2, MBNL1, JADE2, MIF, SLC5A6, C2CD2, CRTAP, ATF1, PPIE, and ADCK3. In some embodiments, the modulation is upmodulation or downmodulation as described in Gene Directionality column of Table 1.

In some embodiments, one or more genes of network module 5 are modulated. In some embodiments, the present disclosure relates to the activation of network module 5, e.g., one or more of (inclusive of all of) SOX4, BAMBI, HES1, ATP1B1, TIMM9, MYC, MAP3K4, CHERP, TBP, DAG1, TEX10, BAG3, NET1, FZD7, RAD9A, NUDT9, and PIK3R4. In some embodiments, the modulation is upmodulation or downmodulation as described in Gene Directionality column of Table 1.

In some embodiments, one or more genes of network module 6 are modulated. In some embodiments, the present disclosure relates to the activation of network module 6, e.g., one or more of (inclusive of all of) DRAP1, SLC25A4, MRPL12, FPGS, ANXA7, HN1L, METRN, LYN, TGFBR2, STAT5B, RAC2, MALT1, DHX29, EPHB2, CDC25B, and PIH1D1. In some embodiments, the modulation is upmodulation or downmodulation as described in Gene Directionality column of Table 1.

In some embodiments, one or more genes of network module 8 are modulated. In some embodiments, the present disclosure relates to the activation of network module 8, e.g., one or more of (inclusive of all of) CDK4, GTPBP8, RBM15B, ELOVL6, IKBKAP, SLC25A13, HSPD1, TSEN2, HEATR1, ME2, BACE2, RFX5, BDH1, PPARG, and SLC37A4. In some embodiments, the modulation is upmodulation or downmodulation as described in Gene Directionality column of Table 1.

In some embodiments, one or more genes of network module 9 are modulated. In some embodiments, the present disclosure relates to the activation of network module 9, e.g., one or more of (inclusive of all of) SMARCD2, NNT, DNM1, ICMT, ETFB, NCK2, and CCND1. In some embodiments, the modulation is upmodulation or downmodulation as described in Gene Directionality column of Table 1.

In some embodiments, the present methods alter a gene signature in the sample of cells, comprising an activation of a network module designated in the network module column of Table 1.

In some embodiments, the activation of the network module designated in the network module column of Table 1 comprises modulating expression and/or activity of 2 or more genes within a network module.

In some embodiments, the activation of the network module designated in the network module column of Table 1 comprises modulating expression and/or activity of all of the genes within a network module.

In some embodiments, the activation of the network module designated in the network module column of Table 1 comprises modulating expression and/or activity of 2 or more genes within 2 or more network modules. In some embodiments, the activation of the network module designated in the network module column of Table 1 comprises modulating expression and/or activity of 2 or more genes (e.g., 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, 50 or more genes, 51 or more, 52 or more, 53 or more, 54 or more, 55 or more, 56 or more, 57 or more, 58 or more, 59 or more, 60 or more, 61 or more, 62 or more, 63 or more, 64 or more, 65 or ore, 66 or more, 67 or more, 68 or more, 69 or more, 70 or more, 71 or more, 72 or more, 73 or more, or 74 genes) within 2 or more network modules (e.g. 2 or more, or 3 or more, or 4 or more, or 5 network modules).

At the time of filing the present disclosure, the World Wide Web at ncbi.nlm.nih.gov/gene provides a description of and the nucleic acid sequence for each Gene designated as an “up” gene in the gene directionality column of Table 1; the contents of each of which is incorporated herein by reference in its entirety. At the time of filing the present disclosure, the World Wide Web at ncbi.nlm.nih.gov/gene provides a description of and the nucleic acid sequence for each Gene listed in the genes designated as a “down” gene in the gene directionality column of Table 1; the contents of each of which is incorporated herein by reference in its entirety.

Perturbagens

A perturbagen useful in the present disclosure can be a small molecule, a biologic, a protein, a nucleic acid, such as a cDNA over-expressing a wild-type gene or an mRNA encoding a wild-type gene, or any combination of any of the foregoing. Illustrative perturbagens useful in the present disclosure and capable of promoting intestinal stem cells or progeny thereof are listed in Table 2.

TABLE 2

| | | Molecular | | |
|---|---|---|---|---|
| Perturbagen | | Weight | | |
| No. | Molecular Formula | (g/mol) | Dose | |
| 1 | C₄₄H₄₇F₂N₉O₅S | 852 | 500 | nM |
| 2 | C₂₂H₂₅NO₆ | 399.4 | 10.0 | μM |
| 3 | C₁₉H₂₈O₄ | 320.4 | 10 | μM |
| 4 | C₂₀H₂₄O₆ | 360.4 | 500 | nM |
| 5 | C₂₉H₃₇N₅O₃ | 503.6 | 0.04 | μM |
| 6 | C₁₅H₁₅FIN₃O₃ | 431.2 | 3.33 | μM |
| 7 | C₁₆H₁₄BrN₅O₄S | 452.3 | 100 | nM |
| 8 | C₂₁H₂₀F₃N₇O₃S | 507.5 | 10 | μM |
| 9 | C₂₁H₂₀N₄O₃ | 376.4 | 10.0 | μM |
| 10 | C₁₈H₁₈CIN₅O | 355.8 | 0.37 | μM |
| 11 | C₂₃H₃₈N₂O₃ | 390.6 | 60 | μM |
| 12 | C₁₇H₁₆N₂O₃ | 296.32 | 10 | μM |

In various embodiments herein, a perturbagen of Table 2 encompasses the perturbagens named in Table 2. Thus, the named perturbagens of Table 2 represent examples of perturbagens of the present disclosure.

In Table 2, the effective in vitro concentration is the exemplary, non-limiting concentration of a perturbagen that is capable of increasing gene expression in a progenitor cell, as assayed, at least, by single cell gene expression profiling (GEP). Although the concentrations were determined in an in vitro assay, the concentrations may be relevant to a determination of in vivo dosages and such dosages may be used in clinic or in clinical testing.

In embodiments, a perturbagen used in the present disclosure is a variant of a perturbagen of Table 2. A variant may be a derivative, analog, enantiomer or a mixture of enantiomers thereof or a pharmaceutically acceptable salt, solvate, hydrate, co-crystal, clathrate, or polymorph of the perturbagen of Table 2. A variant of a perturbagen of Table 2 retains the biological activity of the perturbagen of Table 2.

Methods and Perturbagens for Directing a Change in Cell State

Particular cellular changes in cell state can be matched to differential gene expression (which collectively define a gene signature), caused by exposure of a cell to a perturbagen. In embodiments, a change in cell state may be from one progenitor cell type to another progenitor cell type. In embodiments, a change in cell state may be from an upstream progenitor cell (e.g. early common progenitor) to a downstream progenitor cell. Lastly, in embodiments, a change in cell state may be from the final non-differentiated cell into a differentiated cell.

In some aspects, the present disclosure is related to a method for directing a change in cell state of a progenitor cell. This method includes a step of contacting a population of cells comprising a progenitor cell with at least one perturbagen selected from Table 2, or a variant thereof wherein the at least one perturbagen is capable of altering a gene signature in the progenitor cell; and wherein the progenitor cell is an intestinal stem cell. In some embodiments, the intestinal stem cell can be identified using a LGR5 marker (see Yin et al., Nat Methods. 2014 January; 11(1):106-12).

In aspects, the present disclosure is related to a method for directing a change in cell state of a progenitor cell, comprising contacting a population of cells comprising a progenitor cell with at least one perturbagen capable of altering a gene signature in the progenitor cell; wherein altering the gene signature comprises an increase in expression and/or activity in the progenitor cell of one or more genes designated as an “up” gene in the gene directionality column of Table 1 and/or a decrease in expression and/or activity in the progenitor cell of one or more genes designated as a “down” gene in the gene directionality column of Table 1; and wherein the progenitor cell is an intestinal stem cell.

In some aspects, the present disclosure is related to a method for directing a change in cell state of a progenitor cell, comprising contacting a population of cells comprising a progenitor cell with at least one perturbagen selected from Table 2, or a variant thereof, and capable of altering a gene signature in the progenitor cell; wherein altering the gene signature comprises an increase in expression and/or activity in the progenitor cell of one or more genes designated as an “up” gene in the gene directionality column of Table 1 and/or a decrease in expression and/or activity in the progenitor cell of one or more genes designated as a “down” gene in the gene directionality column of Table 1; and wherein the progenitor cell is an intestinal stem cell.

In some embodiments, the gene signature comprises a change in expression and/or activity of one or more genes in the progenitor cell of a network module designated in the network module column of Table 1. In some embodiments, the activation of one or more genes of the network module designated in the network module column of Table 1 comprises modulating expression and/or activity of 2 or more genes within a network module.

In some embodiments, the step of altering the gene signature comprises an increase in expression and/or activity in the progenitor cell of one or more genes designated as an “up” gene in the gene directionality column of Table 1. In other embodiments, the step of altering the gene signature comprises a decrease in expression and/or activity in the progenitor cell of one or more genes designated as a “down” gene in the gene directionality column of Table 1.

For details on the enteroendocrine cell lineage, see, e.g., Mills, Jason C., and Jeffrey I. Gordon. “The intestinal stem cell niche: there grows the neighborhood.” Proceedings of the National Academy of Sciences 98.22 (2001): 12334-12336, the entire contents of which are incorporated by reference.

In some embodiments, the change in cell state provides an increase in the number of one or more of enteroendocrine cells, goblet progenitors, goblet cells, and Paneth cells. In other embodiments, the change in cell state provides an increase in the number of enteroendocrine cells.

In some embodiments, the increase in the number of enteroendocrine cells is relative to the number of enteroendocrine cells obtained from a population of progenitor cells that is not contacted with the at least one perturbagen. In embodiments, the increase in the number of enteroendocrine cells is relative to the number of enteroendocrine cells obtained from a population of progenitor cells prior to contacting with the at least one perturbagen.

In some embodiments, the change in cell state does not provide a substantial increase in the number of enterocytes and/or provides a decrease in the number of enterocytes.

In some embodiments, the ratio of the number of enteroendocrine cells to the number of enterocytes is increased relative to the ratio obtained from a population of progenitor cells that is not contacted with the at least one perturbagen. In embodiments, the ratio of the number of enteroendocrine cells to the number of enterocytes is increased relative to the ratio in the population of progenitor cells prior to contacting with the at least one perturbagen. In embodiments, the ratio of the number of enteroendocrine cells to the number of progenitor cells is increased relative to the ratio obtained from a population of progenitor cells that is not contacted with the at least one perturbagen. In some embodiments, the ratio of the number of enteroendocrine cells to the number of progenitor cells is increased relative to the ratio in the population of progenitor cells prior to contacting with the at least one perturbagen.

In some embodiments, the methods described herein cause an increase in the number of enteroendocrine cells which, e.g., is due in part to increased cell proliferation of the enteroendocrine cells. In some embodiments, the increase in the number of enteroendocrine cells is due in part to an increased lifespan of the enteroendocrine cells. In other embodiments, the increase in the number of enteroendocrine cells is due in part to reduced cell death among the enteroendocrine cells.

In some embodiments, the methods described herein are such that the number of progenitor cells is decreased. In some embodiments, the decrease in the number of progenitor cells is due in part to decreased cell proliferation of the progenitor cells. In other embodiments, the decrease in the number of progenitor cells is due in part to a decreased lifespan of the progenitor cells. In embodiments, the decrease in the number of progenitor cells is due in part to increased cell death among the progenitor cells.

In some embodiments, the decrease in the number of progenitor cells is relative to the number of progenitor cells in a population of progenitor cells that is not contacted with the at least one perturbagen. In embodiments, the decrease in the number of progenitor cells is relative to the number of progenitor cells in the population prior to contacting with the at least one perturbagen. In some embodiments, the decrease in the number of progenitor cells is due to a change of cell state from a progenitor cell into an enteroendocrine cell.

In some embodiments, the methods described herein are such that the number of progenitor cells is increased. In embodiments, the increase in the number of progenitor cells is due in part to increased cell proliferation of the progenitor cells. In some embodiments, the increase in the number of progenitor cells is due in part to an increased lifespan of the progenitor cells. In embodiments, the increase in the number of progenitor cells is due in part to decreased cell death among the progenitor cells. In embodiments, the increase in the number of progenitor cells is relative to the number of progenitor cells in a population of progenitor cells that is not contacted with the at least one perturbagen. In some embodiments, the increase in the number of progenitor cells is relative to the number of progenitor cells in the population prior to contacting with the at least one perturbagen.

In some embodiments, the methods described herein are such that the number of goblet progenitors, goblet cells, Paneth cells, and/or enteroendocrine cells is increased after contacting the population of cells comprising a progenitor cell with the at least one perturbagen. In some embodiments, the methods described herein are such that the number of goblet progenitors and/or enteroendocrine cells is increased after contacting the population of cells comprising a progenitor cell with the at least one perturbagen. In embodiments, the number of Paneth cells and/or goblet cells is increased after contacting the population of cells comprising a progenitor cell with the at least one perturbagen. In embodiments, the number of enteroendocrine cells is increased after contacting the population of cells comprising a progenitor cell with the at least one perturbagen.

In some embodiments, the methods described herein are such that the ratio of the number of goblet progenitors, goblet cells, Paneth cells, and/or enteroendocrine cells to the number of progenitor cells is increased relative to the ratio obtained from a population of progenitor cells that is not contacted with the at least one perturbagen. In other embodiments, the ratio of the number goblet progenitors, goblet cells, Paneth cells, and/or enteroendocrine cells to the number of progenitor cells is increased relative to the ratio in the population of progenitor cells prior to contacting with the at least one perturbagen. In some embodiments, the ratio of the number of goblet progenitors to the number of progenitor cells is increased relative to the ratio obtained from a population of progenitor cells that is not contacted with the at least one perturbagen.

In embodiments, the ratio of the number of goblet progenitors to the number of progenitor cells is increased relative to the ratio in the population of progenitor cells prior to contacting with the at least one perturbagen. In embodiments, the ratio of the number of goblet progenitors to the number of enterocyte progenitor cells is increased relative to the ratio obtained from a population of progenitor cells that is not contacted with the at least one perturbagen.

In some embodiments, the ratio of the number of goblet progenitors to the number of enterocyte progenitor cells is increased relative to the ratio in the population of progenitor cells prior to contacting with the at least one perturbagen. In embodiments, the ratio of the number of goblet progenitors to the number of enterocytes is increased relative to the ratio obtained from a population of progenitor cells that is not contacted with the at least one perturbagen.

According to some embodiments, the ratio of the number of goblet progenitors to the number of enterocytes is increased relative to the ratio in the population of progenitor cells prior to contacting with the at least one perturbagen. In some embodiments, the ratio of the number of enteroendocrine cells to the number of goblet progenitors is increased relative to the ratio obtained from a population of progenitor cells that is not contacted with the at least one perturbagen. In some embodiments, the ratio of the number of enteroendocrine cells to the number of goblet progenitors is increased relative to the ratio in the population of progenitor cells prior to contacting with the at least one perturbagen. In some embodiments, the ratio of the number of enteroendocrine cells to the number of intestinal stem cells is increased relative to the ratio obtained from a population of progenitor cells that is not contacted with the at least one perturbagen.

According to some embodiments, the ratio of the number of enteroendocrine cells to the number of intestinal stem cells is increased relative to the ratio in the population of progenitor cells prior to contacting with the at least one perturbagen. In some embodiments, the ratio of the number of enteroendocrine cells to the number of Paneth cells is increased relative to the ratio obtained from a population of progenitor cells that is not contacted with the at least one perturbagen. In embodiments, the ratio of the number of enteroendocrine cells to the number of Paneth cells is increased relative to the ratio in the population of progenitor cells prior to contacting with the at least one perturbagen. In some embodiments, the ratio of the number of enteroendocrine cells to the number of enteroendocrine cells is increased relative to the ratio obtained from a population of progenitor cells that is not contacted with the at least one perturbagen. In embodiments, the ratio of the number of enteroendocrine cells to the number of goblet cells is increased relative to the ratio obtained from a population of progenitor cells that is not contacted with the at least one perturbagen. In embodiments, the ratio of the number of enteroendocrine cells to the number of goblet cells is increased relative to the ratio in the population of progenitor cells prior to contacting with the at least one perturbagen. In some embodiments, the ratio of the number of enteroendocrine cells to the number of enterocyte progenitor cells is increased relative to the ratio obtained from a population of progenitor cells that is not contacted with the at least one perturbagen. According to some embodiments, the ratio of the number of enteroendocrine cells to the number of enterocyte progenitor cells is increased relative to the ratio in the population of progenitor cells prior to contacting with the at least one perturbagen.

In some embodiments, the ratio of the number of goblet cells, Paneth cells, and enteroendocrine cells to the number of enterocytes is increased relative to the ratio obtained from a population of progenitor cells that is not contacted with the at least one perturbagen. In some embodiments, the ratio of the number of goblet cells, Paneth cells, and enteroendocrine cells to the number of enterocytes is increased relative to the ratio in the population of progenitor cells prior to contacting with the at least one perturbagen.

According to some embodiments, the number of goblet progenitors, Paneth cells, goblet cells, enterocyte progenitor cells, and/or enterocytes is decreased. In embodiments, the number of goblet progenitors, goblet cells, Paneth cells, enterocyte progenitor cells, and/or enterocytes is decreased. In some embodiments, the number of goblet progenitors is decreased. In embodiments, the number of Paneth cells is decreased. In some embodiments, the number of goblet cells is decreased. In embodiments, the number of enterocyte progenitor cells is decreased. In some embodiments, the number of enterocytes is decreased.

According to some embodiments, the methods described herein are such that the number of goblet progenitors, goblet cells, Paneth cells, and/or enteroendocrine cells is increased. In some embodiments, the number of goblet progenitors is increased. In some embodiments, the number of goblet cells is increased. In other embodiments, the number of Paneth cells is increased. In some embodiments, the number of enteroendocrine cells is increased.

In some embodiments, the methods described here use at least one perturbagen selected from Table 2, or a variant thereof. In some embodiments, the at least one perturbagen comprises at least 2, at least 3, at least 4, at least 5, at least 6, at least 7, at least 8, at least 9, at least 10, at least 11, or all 12 perturbagens selected from Table 2, or variants thereof.

In some embodiments, the one or more genes designated as an “up” gene in the gene directionality column of Table 1 comprises 2 or more, 3 or more, 4 or more, 5 or more, 6 or more, or 7 genes. In embodiments, the one or more genes designated as an “up” gene in the gene directionality column of Table 1 comprises at least one of NPDC1, SOX4, BAMBI, DRAP1, SLC25A4, CDK4, and SMARCD2.

In some embodiments, the one or more genes designated as a “down” gene in the gene directionality column of Table 1 comprise 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, or 68 genes. In embodiments, the one or more genes designated as a “down” gene in the gene directionality column of Table 1 comprise at least one of CD44, DCTD, CDK6, GAA, DDB2, HMGA2, ST7, SLC35F2, MLEC, DPH2, MBNL1, JADE2, MIF, SLC5A6, C2CD2, CRTAP, ATF1, PPIE, ADCK3, HES1, ATP1B1, TIMM9, MYC, MAP3K4, CHERP, TBP, DAG1, TEX10, BAG3, NET1, FZD7, RAD9A, NUDT9, PIK3R4, MRPL12, FPGS, ANXA7, HN1L, METRN, LYN, TGFBR2, STAT5B, RAC2, MALT1, DHX29, EPHB2, CDC25B, PIH1D1, GTPBP8, RBM15B, ELOVL6, IKBKAP, SLC25A13, HSPD1, TSEN2, HEATR1, ME2, BACE2, RFX5, BDH1, PPARG, SLC37A4, NNT, DNM1, ICMT, ETFB, NCK2, and CCND1.

In embodiments, an increase in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more decrease in gene expression relative to a cell that has not been contacted with a perturbagen and/or relative to a cell that has been contacted with a no treatment control (including DMSO).

In various embodiments, an increase in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater decrease in gene expression relative to a cell that has not been contacted with a perturbagen and/or relative to a cell that has been contacted with a no treatment control (including DMSO).

In embodiments, contacting the population of cells comprising a progenitor cell occurs in vitro or ex vivo.

In embodiments, contacting the population of cells comprising a progenitor cell occurs in vivo in a subject. In embodiments, the subject is a human. In embodiments, the human is an adult human.

In some embodiments, the change in cells state provides increased secretion of cholecystokinin (CCK), glucagon-like peptide 1 and 2 (GLP-1 and GLP-2), glucose dependent insulinotropic peptide (GIP), peptide YY (PYY), gastrin, secretin, somatostatin, motilin, leptin, nesfatin-1, and ghrelin, bioactive amines, histamine, serotonin (5-HT), neurotensin, vasoactive intestinal peptide, and enteroglucagon by an enteroendocrine cell.

In some embodiments, various subtypes of enteroendocrine cells are influenced by the methods described herein, e.g. any subtype of enteroendocrine cell is increased in numbers or as a ratio to other cells. Various subtypes of enteroendocrine cells include: A (X-like) cells (Ghrelin, nesfatin-1), Enterochromaffin-like-cell (histamine), G cells (Gastrin), P cells (Leptin), D cells (Somatostatin) Enterochromaffin cell (5-HT), I cells (CCK), K cells (GIP), S cells (Secretin) M cells (Motilin), L cells (GLP-1, GLP-2, PYY, oxyntomodulin), N cells (Neurotensin). The enteroendocrine cells described herein have one or more functions selected from: enabling efficient post-prandial assimilation of nutrients via alterations in gastrointestinal secretion, motility, pancreatic insulin release, satiety, regulating energy homeostasis, glucose metabolism, gut barrier function, and/or mucosal immunity.

Various cells markers associates with the enteroendocrine cells include, e.g., Neurogenin 3 (Neurog3), micro-RNA-375, neurogenic differentiation 1 (Neurod1), Pax4, Pax 66, insulin gene enhancer protein (Isl1), pancreatic and duodenal homeobox 1 (Pdx1), Nkx6-1, Nkx2-2, caudal type homeobox 2 (Cdx-2), Gata4, Gata-5, Gata6, hepatocyte nuclear factor-1a (Hnf-1a), Hnf-1b, CCAAT-displacement protein (Cdp), chromogranin A, chromogranin B, Ghrelin, nesfatin-1, histamine, Gastrin, Leptin, Somatostatin, 5-HT, CCK, GIP, Secretin, Motilin, GLP-1, GLP-2, PYY, oxyntomodulin, and/or Neurotensin.

In yet another aspect, the present disclosure provides a perturbagen for use in any herein disclosed method.

In a further aspect, the present disclosure provides a pharmaceutical composition comprising perturbagen for use in any herein disclosed method.

In some aspects, the present disclosure is related to a method for promoting the formation of a enteroendocrine cells, or an immediate progenitor thereof, comprising: exposing a starting population of intestinal stem cells to a perturbation having a perturbation signature that promotes the transition of the starting population of intestinal stem cells into a goblet progenitor cell or an enteroendocrine cell, wherein the perturbation signature comprises increased expression and/or activity in the intestinal stem cell of one or more of genes designated as an “up” gene in the gene directionality column of Table 1 and/or a decreased expression and/or activity in the intestinal stem cell of one or more genes designated as a “down” gene in the gene directionality column of Table 1.

In some embodiments, the perturbation signature comprises an increase in expression and/or activity of one or more genes in the progenitor cell of an activation of a network module designated in the network module column of Table 1. In embodiments, the activation of one or more genes of the network module designated in the network module column of Table 1 comprises modulating expression and/or activity of 2 or more genes within a network module.

In some embodiments, the perturbation signature comprises an increase in expression and/or activity in the progenitor cell of two or more genes designated as an “up” gene in the gene directionality column of Table 1. In embodiments, the perturbation signature comprises a decrease in expression and/or activity in the progenitor cell of two or more genes designated as a “down” gene in the gene directionality column of Table 1.

Embodiments associated with the above aspects are likewise relevant to the present aspect. In other words, each of the embodiments mentioned above for the above aspects may be revised/adapted to be applicable to the present aspect.

Methods and Perturbagens for Treating a Disease or Disorder

The ability of a perturbagen to specifically promote one or more of goblet progenitors, goblet cells, Paneth cells, and/or enteroendocrine cells would be valuable in designing a therapeutic composition. As examples, for a disease characterized by a reduced number of one or more of goblet progenitors, goblet cells, Paneth cells, and/or enteroendocrine cells, a therapeutic composition comprising a perturbagen that increases the number of one or more of goblet progenitors, goblet cells, Paneth cells, and/or enteroendocrine cells could be beneficial. A disease that would benefit from increased numbers of goblet progenitors or enteroendocrine cells could be treated by a therapeutic composition comprising a perturbagen that increases the number of goblet progenitors or enteroendocrine cells.

Another aspect of the present disclosure is related to a method for treating a disease or disorder characterized by an abnormal number or abnormal function of enteroendocrine cells, comprising: (a) administering to a patient in need thereof a therapeutically effective amount of at least one perturbagen selected from Table 2, or a variant thereof, wherein the at least one perturbagen is capable of changing a gene signature in a progenitor cell, or (b) administering to a patient in need thereof a cell, the cell having been contacted with at least one perturbagen selected from Table 2, or a variant thereof, wherein the at least one perturbagen is capable of changing a gene signature in a progenitor cell.

In some embodiments, the disease or disorder is caused by an enteroendocrine cell deficiency.

In some embodiments, the at least one perturbagen is administered on the basis of previously determining that the patient exhibits an abnormal number or abnormal function of enteroendocrine cells, or a disease or disorder characterized thereby. In embodiments, the administering, as described herein, is directed to the bone marrow of the patient. In embodiments, the administering is via intraosseous injection or intraosseous infusion. In embodiments, the administering the cell is via intravenous injection or intravenous infusion. In other embodiments, the administering of the cell is via intravenous injection or intravenous infusion. In some embodiments, the administering is simultaneously or sequentially to one or more mobilization agents. In some embodiments, the administering of the perturbagen is via oral, intravenous, intramuscular, intraperitoneal, subcutaneous, intra-articular injection, and/or infusion route. In embodiments, the delivery/administration of the perturbagen is via the gastrointestinal (GI) tract, optionally selected from the stomach, small intestine, duodenum, jejunum, ileum, large intestine, colon transversum, colon descendens, colon ascendens, colon sigmoidenum, cecum, and rectum.

In some embodiments, the perturbagen is formulated with a delayed-release coating, which is optionally enzyme-dependent.

In some embodiments, the disease or disorder that is treated by the methods described herein is selected from Type II Diabetes, obesity, weight loss, intestinal inflammation (e.g. inflammatory bowel disease, infection, colorectal cancer or food allergies), nonalcoholic fatty liver disease, and cardiovascular complications of diabetes. In some embodiment, the at least one perturbagen is administered on the basis of previously determining that the patient exhibits an abnormal number of enteroendocrine cells, or a disease or disorder characterized thereby.

Yet another aspect of the present disclosure is related to a method for treating a disease or disorder characterized by an abnormal ratio of enteroendocrine cells to intestinal stem cells, comprising: (a) administering to a patient in need thereof at least one perturbagen selected from Table 2, or a variant thereof, wherein the at least one perturbagen is capable of changing a gene signature in an intestinal stem cell or (b) administering to a patient in need thereof a cell, the cell having been contacted with at least one perturbagen selected from Table 2, or a variant thereof, wherein the at least one perturbagen is capable of changing a gene signature in an intestinal stem cell. In some embodiments, the abnormal ratio comprises a decreased number of enteroendocrine cells and/or an increased number of intestinal stem cells. In embodiments, the abnormal ratio comprises an increased number of intestinal stem cells. In other embodiments, the abnormal ratio comprises a decreased number of enteroendocrine cells.

In some embodiments, the administering according to this aspect is directed to the bone marrow of the patient. In embodiments, the administering is via intraosseous injection or intraosseous infusion. In some embodiments, the administering the cell is via intravenous injection or intravenous infusion. In some embodiments, the administering is simultaneously or sequentially to one or more mobilization agents. In embodiments, the administering of the perturbagen is via oral, intravenous, intramuscular, intraperitoneal, subcutaneous, intra-articular injection, and/or infusion route. In some embodiments, the delivery/administration of the perturbagen is via the gastrointestinal (GI) tract, optionally selected from the stomach, small intestine, duodenum, jejunum, ileum, large intestine, colon transversum, colon descendens, colon ascendens, colon sigmoidenum, cecum, and rectum.

In some embodiments, the disease or disorder that is treated by according to this aspect of the disclosure is selected from Type II Diabetes, obesity, weight loss, intestinal inflammation (e.g. inflammatory bowel disease, infection, colorectal cancer or food allergies), nonalcoholic fatty liver disease, and cardiovascular complications of diabetes.

In some embodiments, the at least one perturbagen is administered on the basis of previously determining that the patient exhibits the abnormal ratio of enteroendocrine cells to intestinal stem cells, or a disease or disorder characterized thereby. In embodiments, the at least one perturbagen is capable of changing a gene signature in a progenitor cell.

In some embodiments, the present disclosure is related to selection of a patient. The patient is selected by steps comprising: obtaining from the patient having the disease or disorder a sample of cells comprising at least one intestinal stem cell; and contacting the sample of cells with least one perturbagen selected from Table 2, or a variant thereof, wherein the at least one perturbagen alters a gene signature in the sample of cells.

In some embodiments, the patient is selected by steps comprising: obtaining from a subject having the disease or disorder a sample of cells comprising at least one intestinal stem cell; and contacting the sample of cells with at least one perturbagen capable of altering a gene signature in an intestinal stem cell, wherein the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1. In some embodiments, the perturbagen causes an increase in expression and/or activity of one or more genes in the progenitor cell of an activation of a network module designated in the network module column of Table 1. In some embodiments, the activation of one or more genes of the network module designated in the network module column of Table 1 comprises modulating expression and/or activity of 2 or more genes within a network module. In some embodiments, the perturbagen causes an increase in expression and/or activity in the progenitor cell of two or more genes designated as an “up” gene in the gene directionality column of Table 1. In some embodiments, the perturbagen causes a decrease in expression and/or activity in the progenitor cell of two or more genes designated as a “down” gene in the gene directionality column of Table 1.

In embodiments, the patient is selected by steps comprising: obtaining from a subject having the disease or disorder a sample of cells comprising an intestinal stem cell; and contacting the sample of cells with at least one perturbagen selected from Table 2, or a variant thereof; wherein the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1. In some embodiments, the perturbagen causes an increase in expression and/or activity of one or more genes in the progenitor cell of an activation of a network module designated in the network module column of Table 1. In some embodiments, the activation of one or more genes of the network module designated in the network module column of Table 1 comprises modulating expression and/or activity of 2 or more genes within a network module. In embodiments, the perturbagen causes an increase in expression and/or activity in the progenitor cell of two or more genes designated as an “up” gene in the gene directionality column of Table 1. In some embodiments, the perturbagen causes a decrease in expression and/or activity in the progenitor cell of two or more genes designated as a “down” gene in the gene directionality column of Table 1.

In some embodiments, the present disclosure is related to a method for selecting the patient comprising: obtaining from a subject having the disease or disorder a sample of cells comprising an intestinal stem cell; and contacting the sample of cells with least one perturbagen selected from Table 2, or a variant thereof, wherein when the at least one perturbagen alters a gene signature in the sample of cells, the subject is selected as a patient.

In some embodiments, the method for selecting the patient according to any of the methods described herein include a step of selecting a patient that includes obtaining from a subject, having the disease or disorder described herein, a sample of cells comprising an intestinal stem cell; and contacting the sample of cells with at least one perturbagen capable of altering a gene signature in an intestinal stem cell, wherein when the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1, the subject is selected as a patient. In some embodiments, altering the gene signature comprises a change in expression and/or activity of one or more genes in the progenitor cell of a network module designated in the network module column of Table 1. In embodiments, the activation of one or more genes of the network module designated in the network module column of Table 1 comprises modulating expression and/or activity of 2 or more genes within a network module. In some embodiments, altering the gene signature comprises an increase in expression and/or activity in the progenitor cell of two or more genes designated as an “up” gene in the gene directionality column of Table 1. In some embodiments, altering the gene signature comprises a decrease in expression and/or activity in the progenitor cell of two or more genes designated as a “down” gene in the gene directionality column of Table 1.

In some embodiments, the method for selecting the patient according to any of the methods described herein include obtaining from a subject, having the disease or disorder described herein, a sample of cells comprising an intestinal stem cell; and contacting the sample of cells with at least one perturbagen selected from Table 2, or a variant thereof; wherein when the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1, the subject is selected as a patient. In some embodiments, the perturbagen causes an increase in expression and/or activity of one or more genes in the progenitor cell of an activation of a network module designated in the network module column of Table 1. In some embodiments, the activation of one or more genes of the network module designated in the network module column of Table 1 comprises modulating expression and/or activity of 2 or more genes within a network module. In embodiments, the perturbagen causes an increase in expression and/or activity in the progenitor cell of two or more genes designated as an “up” gene in the gene directionality column of Table 1. In some embodiments, the perturbagen causes a decrease in expression and/or activity in the progenitor cell of two or more genes designated as a “down” gene in the gene directionality column of Table 1.

In some embodiments, the present disclosure is related to the use of the perturbagen of Table 2, or a variant thereof in the manufacture of a medicament for treating a disease or disorder characterized by an abnormal ratio of enteroendocrine cells to intestinal stem cells. In embodiments, the present disclosure is related to the use of the perturbagen of Table 2, or a variant thereof in the manufacture of a medicament for treating a disease or disorder characterized by an abnormal ratio of enteroendocrine cells to enterocytes, Paneth cells and/or goblet cells.

In some aspects, the present disclosure is related to a method of identifying a candidate perturbation for promoting the transition of a starting population of intestinal stem cells into enteroendocrine cells or immediate progenitors thereof, the method comprising: exposing the starting population of intestinal stem cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of intestinal stem cells into enteroendocrine cells or immediate progenitors thereof following exposure of the population of cells to the perturbation; and identifying the perturbation as a candidate perturbation for promoting the transition of a population of intestinal stem cells into enteroendocrine cells or immediate progenitors thereof based on the perturbation signature, wherein the perturbation signature is an increase in expression and/or activity in the intestinal stem cell of one or more genes designated as an “up” gene in the gene directionality column of Table 1, and/or a decrease in expression and/or activity in the intestinal stem cell of one or more genes designated as a “down” gene in the gene directionality column of Table 1. In some embodiments, the perturbation signature is an increase in expression and/or activity in the progenitor cell of an activation of a network module designated in the network module column of Table 1. In some embodiments, the activation of one or more genes of the network module designated in the network module column of Table 1 comprises modulating expression and/or activity of 2 or more genes within a network module. In embodiments, the perturbation signature is an increase in expression and/or activity in the progenitor cell of two or more genes designated as an “up” gene in the gene directionality column of Table 1. In some embodiments, the perturbation signature is a decrease in expression and/or activity in the progenitor cell of two or more genes designated as a “down” gene in the gene directionality column of Table 1.

In some aspects, the present disclosure is related to a method for making a therapeutic agent for a disease or disorder selected from Type II Diabetes, obesity, weight loss, intestinal inflammation (e.g. inflammatory bowel disease, infection, colorectal cancer or food allergies), nonalcoholic fatty liver disease, and cardiovascular complications of diabetes, comprising: (a) identifying a therapeutic agent for therapy according to methods described herein and (b) formulating the therapeutic agent for the treatment of the disease or disorder.

Methods for determining the extension of the lifespan of a specific cell type or a reduction of cell death is well known in the art. As examples, markers for dying cells, e.g., caspases can be detected, or dyes for dead cells, e.g., methylene blue, may be used. Methods for counting cells are well known in the art. Non-limiting examples include hemocytometry, flow cytometry, and cell sorting techniques, e.g., fluorescence activated cell sorting (FACS).

Administration, Dosing, and Treatment Regimens

As examples, administration results in the delivery of one or more perturbagens disclosed herein into the bloodstream (via enteral or parenteral administration), or alternatively, the one or more perturbagens is administered directly to the site of stem cell proliferation and/or maturation, i.e., in the bone marrow.

Delivery of one or more perturbagens disclosed herein to the bone marrow may be via intravenous injection or intravenous infusion or via intraosseous injection or intraosseous infusion. Devices and apparatuses for performing these delivery methods are well known in the art.

Delivery of one or more perturbagens disclosed herein into the bloodstream via intravenous injection or intravenous infusion may follow or be contemporaneous with stem cell mobilization. In stem cell mobilization, certain drugs are used to cause the movement of stem cells from the bone marrow into the bloodstream. Once in the bloodstream, the stem cells are contacted with the one or more perturbagens and are able to alter a gene signature in a progenitor cell, for example. Drugs and methods relevant to stem cell mobilization are well known in the art; see, e.g., Mohammadi et al, “Optimizing Stem Cells Mobilization Strategies to Ameliorate Patient Outcomes: A Review of Guide-lines and Recommendations.” Int. J. Hematol. Oncol. Stem Cell Res. 2017 Jan. 1; 11(1): 78-88; Hopman and DiPersio “Advances in Stem Cell Mobilization.” Blood Review, 2014, 28(1): 31-40; and Kim “Hematopoietic stem cell mobilization: current status and future perspective.” Blood Res. 2017 June; 52(2): 79-81. The content of each of which is incorporated herein by reference in its entirety.

Dosage forms suitable for parenteral administration include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any perturbagen disclosed herein as well as the dosing schedule can depend on various parameters and factors, including, but not limited to, the specific perturbagen, the disease being treated, the severity of the condition, whether the condition is to be treated or prevented, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In another embodiment, delivery can be in a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

A perturbagen disclosed herein can be administered by a controlled-release or a sustained-release means or by delivery a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, e.g., the bone marrow, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533 may be used.

The dosage regimen utilizing any perturbagen disclosed herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the disclosure employed. Any perturbagen disclosed herein can be administered in a single daily dose, or the total daily dosage can be administered in divided doses of two, three or four times daily. Furthermore, any perturbagen disclosed herein can be administered continuously rather than intermittently throughout the dosage regimen.

Pharmaceutical Compositions and Formulations

Aspects of the present disclosure include a pharmaceutical composition comprising a therapeutically effective amount of one or more perturbagens, as disclosed herein.

The perturbagens disclosed herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety. In embodiments, the compositions disclosed herein are in the form of a pharmaceutically acceptable salt.

Further, any perturbagen disclosed herein can be administered to a subject as a component of a composition, e.g., pharmaceutical composition that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any perturbagen disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

In embodiments, the compositions, e.g., pharmaceutical compositions, disclosed herein are suspended in a saline buffer (including, without limitation TBS, PBS, and the like).

The present disclosure includes the disclosed perturbagens in various formulations of pharmaceutical compositions. Any perturbagens disclosed herein can take the form of solutions, suspensions, emulsion, drops, tablets, pills, pellets, capsules, capsules containing liquids, powders, sustained-release formulations, emulsions, aerosols, sprays, suspensions, or any other form suitable for use.

Where necessary, the pharmaceutical compositions comprising the perturbagens can also include a solubilizing agent. Also, the agents can be delivered with a suitable vehicle or delivery device as known in the art.

Combination therapies, comprising more than one perturbagen, can be co-delivered in a single delivery vehicle or delivery device. Compositions for administration can optionally include a local anesthetic such as, for example, lignocaine to lessen pain at the site of the injection.

The pharmaceutical compositions comprising the perturbagens of the present disclosure may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In embodiments, any perturbagens disclosed herein is formulated in accordance with routine procedures as a pharmaceutical composition adapted for a mode of administration disclosed herein.

Yet another aspect of the present disclosure is a perturbagen capable of causing a change in a gene signature.

In an aspect, the present disclosure provides a perturbagen capable of causing a change in cell fate.

In another aspect, the present disclosure provides a perturbagen capable of causing a change in a gene signature and a change in cell fate.

In yet another aspect, the present disclosure provides a pharmaceutical composition comprising any herein disclosed perturbagen.

In a further aspect, the present disclosure provides a unit dosage form comprising an effective amount of the pharmaceutical composition comprising any herein disclosed perturbagen.

The instant disclosure also provides certain embodiments as follows:

Embodiment 1. A method for directing a change in cell state of a progenitor cell comprising: contacting a population of cells comprising a progenitor cell with at least one perturbagen selected from Table 2, or a variant thereof; wherein the at least one perturbagen is capable of altering a gene signature in the progenitor cell; and wherein the progenitor cell is an intestinal stem cell.

Embodiment 2. A method for directing a change in cell state of a progenitor cell, comprising: contacting a population of cells comprising a progenitor cell with at least one perturbagen capable of altering a gene signature in the progenitor cell; wherein altering the gene signature comprises an increase in expression and/or activity in the progenitor cell of one or more genes designated as an “up” gene in the gene directionality column of Table 1 and/or a decrease in expression and/or activity in the progenitor cell of one or more genes designated as a “down” gene in the gene directionality column of Table 1; and wherein the progenitor cell is an intestinal stem cell.

Embodiment 3. A method for directing a change in cell state of a progenitor cell, comprising: contacting a population of cells comprising a progenitor cell with at least one perturbagen selected from Table 2, or a variant thereof, and capable of altering a gene signature in the progenitor cell; wherein altering the gene signature comprises an increase in expression and/or activity in the progenitor cell of one or more genes designated as an “up” gene in the gene directionality column of Table 1 and/or a decrease in expression and/or activity in the progenitor cell of one or more genes designated as a “down” gene in the gene directionality column of Table 1; and wherein the progenitor cell is an intestinal stem cell.

Embodiment 4: The method of any one of Embodiments 1-3, wherein altering the gene signature comprises a change in expression and/or activity of one or more genes in the progenitor cell of a network module designated in the network module column of Table 1.

Embodiment 5. The method of any one of Embodiments 1-4, wherein the activation of one or more genes of the network module designated in the network module column of Table 1 comprises modulating expression and/or activity of 2 or more genes within a network module.

Embodiment 6. The method of any one of Embodiments 1-5, wherein altering the gene signature comprises an increase in expression and/or activity in the progenitor cell of two or more genes designated as an “up” gene in the gene directionality column of Table 1.

Embodiment 7. The method of any one of Embodiments 1-6, wherein altering the gene signature comprises a decrease in expression and/or activity in the progenitor cell of two or more genes designated as a “down” gene in the gene directionality column of Table 1.

Embodiment 8. The method of any one of Embodiments 1 to 3, wherein the change in cell state provides an increase in the number of one or more of enteroendocrine cells, goblet progenitors, goblet cells, and Paneth cells.

Embodiment 9. The method of Embodiment 8, wherein the change in cell state provides an increase in the number of enteroendocrine cells.

Embodiment 10. The method of Embodiment 9, wherein the increase in the number of enteroendocrine cells is relative to the number of enteroendocrine cells obtained from a population of progenitor cells that is not contacted with the at least one perturbagen.

Embodiment 11. The method of Embodiment 9, wherein the increase in the number of enteroendocrine cells is relative to the number of enteroendocrine cells obtained from a population of progenitor cells prior to contacting with the at least one perturbagen.

Embodiment 12. The method of Embodiment 8, wherein the change in cell state does not provide a substantial increase in the number of enterocytes and/or provides a decrease in the number of enterocytes.

Embodiment 13. The method of any one of Embodiments 9 to 11, wherein the ratio of the number of enteroendocrine cells to the number of enterocytes is increased relative to the ratio obtained from a population of progenitor cells that is not contacted with the at least one perturbagen.

Embodiment 14. The method of any one of Embodiments 9 to 11, wherein the ratio of the number of enteroendocrine cells to the number of enterocytes is increased relative to the ratio in the population of progenitor cells prior to contacting with the at least one perturbagen.

Embodiment 15. The method of Embodiment 9, wherein the ratio of the number of enteroendocrine cells to the number of progenitor cells is increased relative to the ratio obtained from a population of progenitor cells that is not contacted with the at least one perturbagen.

Embodiment 16. The method of Embodiment 9, wherein the ratio of the number of enteroendocrine cells to the number of progenitor cells is increased relative to the ratio in the population of progenitor cells prior to contacting with the at least one perturbagen.

Embodiment 17. The method of any one of Embodiments 8 to 11, wherein the increase in the number of enteroendocrine cells is due in part to increased cell proliferation of the enteroendocrine cells.

Embodiment 18. The method of any one of Embodiments 8 to 11, wherein the increase in the number of enteroendocrine cells is due in part to an increased lifespan of the enteroendocrine cells.

Embodiment 19. The method of any one of Embodiments 8 to 11, wherein the increase in the number of enteroendocrine cells is due in part to reduced cell death among the enteroendocrine cells.

Embodiment 20. The method of any one of Embodiments 1 to 19, wherein the number of progenitor cells is decreased.

Embodiment 21. The method of Embodiment 20, wherein the decrease in the number of progenitor cells is due in part to decreased cell proliferation of the progenitor cells.

Embodiment 22. The method of Embodiment 20 or Embodiment 21, wherein the decrease in the number of progenitor cells is due in part to a decreased lifespan of the progenitor cells.

Embodiment 23. The method of any one of Embodiments 20 to 22, wherein the decrease in the number of progenitor cells is due in part to increased cell death among the progenitor cells.

Embodiment 24. The method of any one of Embodiments 20 to 23, wherein the decrease in the number of progenitor cells is relative to the number of progenitor cells in a population of progenitor cells that is not contacted with the at least one perturbagen.

Embodiment 25. The method of any one of Embodiments 20 to 23, wherein the decrease in the number of progenitor cells is relative to the number of progenitor cells in the population prior to contacting with the at least one perturbagen.

Embodiment 26. The method of any one of Embodiments 20 to 23, wherein the decrease in the number of progenitor cells is due to a change of cell state from a progenitor cell into an enteroendocrine cell.

Embodiment 27. The method of any one of Embodiments 1 to 26, wherein the number of progenitor cells is increased.

Embodiment 28. The method of Embodiment 27, wherein the increase in the number of progenitor cells is due in part to increased cell proliferation of the progenitor cells.

Embodiment 29. The method of Embodiment 27 or Embodiment 28, wherein the increase in the number of progenitor cells is due in part to an increased lifespan of the progenitor cells.

Embodiment 30. The method of any one of Embodiments 27 to 29, wherein the increase in the number of progenitor cells is due in part to decreased cell death among the progenitor cells.

Embodiment 31. The method of any one of Embodiments 27 to 30, wherein the increase in the number of progenitor cells is relative to the number of progenitor cells in a population of progenitor cells that is not contacted with the at least one perturbagen.

Embodiment 32. The method of any one of Embodiments 27 to 30, wherein the increase in the number of progenitor cells is relative to the number of progenitor cells in the population prior to contacting with the at least one perturbagen.

Embodiment 33. The method of any one of Embodiments 1 to 19, wherein the number of goblet progenitors, goblet cells, Paneth cells, and/or enteroendocrine cells is increased after contacting the population of cells comprising a progenitor cell with the at least one perturbagen.

Embodiment 34. The method of any one of Embodiments 1 to 19, wherein the number of goblet progenitors and/or enteroendocrine cells is increased after contacting the population of cells comprising a progenitor cell with the at least one perturbagen.

Embodiment 35. The method of any one of Embodiments 1 to 19, wherein the number of Paneth cells and/or goblet cells is increased after contacting the population of cells comprising a progenitor cell with the at least one perturbagen.

Embodiment 36. The method of any one of Embodiments 1 to 19, wherein the number of enteroendocrine cells is increased after contacting the population of cells comprising a progenitor cell with the at least one perturbagen.

Embodiment 37. The method of Embodiment 33, wherein the ratio of the number of goblet progenitors, goblet cells, Paneth cells, and/or enteroendocrine cells to the number of progenitor cells is increased relative to the ratio obtained from a population of progenitor cells that is not contacted with the at least one perturbagen.

Embodiment 38. The method of Embodiment 33, wherein the ratio of the number goblet progenitors, goblet cells, Paneth cells, and/or enteroendocrine cells to the number of progenitor cells is increased relative to the ratio in the population of progenitor cells prior to contacting with the at least one perturbagen.

Embodiment 39. The method of any one of Embodiments 1 to 19, wherein the ratio of the number of goblet progenitors to the number of progenitor cells is increased relative to the ratio obtained from a population of progenitor cells that is not contacted with the at least one perturbagen.

Embodiment 40. The method of Embodiment 39, wherein the ratio of the number of goblet progenitors to the number of progenitor cells is increased relative to the ratio in the population of progenitor cells prior to contacting with the at least one perturbagen.

Embodiment 41. The method of any one of Embodiments 1 to 19, wherein the ratio of the number of goblet progenitors to the number of enterocyte progenitor cells is increased relative to the ratio obtained from a population of progenitor cells that is not contacted with the at least one perturbagen.

Embodiment 42. The method of Embodiment 41, wherein the ratio of the number of goblet progenitors to the number of enterocyte progenitor cells is increased relative to the ratio in the population of progenitor cells prior to contacting with the at least one perturbagen.

Embodiment 43. The method of any one of Embodiments 1 to 19, wherein the ratio of the number of goblet progenitors to the number of enterocytes is increased relative to the ratio obtained from a population of progenitor cells that is not contacted with the at least one perturbagen.

Embodiment 44. The method of Embodiment 43, wherein the ratio of the number of goblet progenitors to the number of enterocytes is increased relative to the ratio in the population of progenitor cells prior to contacting with the at least one perturbagen.

Embodiment 45. The method of any one of Embodiments 1 to 19, wherein the ratio of the number of enteroendocrine cells to the number of goblet progenitors is increased relative to the ratio obtained from a population of progenitor cells that is not contacted with the at least one perturbagen.

Embodiment 46. The method of Embodiment 45, wherein the ratio of the number of enteroendocrine cells to the number of goblet progenitors is increased relative to the ratio in the population of progenitor cells prior to contacting with the at least one perturbagen.

Embodiment 47. The method of any one of Embodiments 1 to 19, wherein the ratio of the number of enteroendocrine cells to the number of intestinal stem cells is increased relative to the ratio obtained from a population of progenitor cells that is not contacted with the at least one perturbagen.

Embodiment 48. The method of Embodiment 47, wherein the ratio of the number of enteroendocrine cells to the number of intestinal stem cells is increased relative to the ratio in the population of progenitor cells prior to contacting with the at least one perturbagen.

Embodiment 49. The method of any one of Embodiments 1 to 19, wherein the ratio of the number of enteroendocrine cells to the number of Paneth cells is increased relative to the ratio obtained from a population of progenitor cells that is not contacted with the at least one perturbagen.

Embodiment 50. The method of Embodiment 49, wherein the ratio of the number of enteroendocrine cells to the number of Paneth cells is increased relative to the ratio in the population of progenitor cells prior to contacting with the at least one perturbagen.

Embodiment 51. The method of any one of Embodiments 1 to 19, wherein the ratio of the number of enteroendocrine cells to the number of goblet cells is increased relative to the ratio obtained from a population of progenitor cells that is not contacted with the at least one perturbagen.

Embodiment 52. The method of Embodiment 51, wherein the ratio of the number of enteroendocrine cells to the number of goblet cells is increased relative to the ratio in the population of progenitor cells prior to contacting with the at least one perturbagen.

Embodiment 53. The method of any one of Embodiments 1 to 19, wherein the ratio of the number of enteroendocrine cells to the number of enterocyte progenitor cells is increased relative to the ratio obtained from a population of progenitor cells that is not contacted with the at least one perturbagen.

Embodiment 54. The method of Embodiment 53, wherein the ratio of the number of enteroendocrine cells to the number of enterocyte progenitor cells is increased relative to the ratio in the population of progenitor cells prior to contacting with the at least one perturbagen.

Embodiment 55. The method of any one of Embodiments 1 to 19, wherein the ratio of the number of goblet cells, Paneth cells, and enteroendocrine cells to the number of enterocytes is increased relative to the ratio obtained from a population of progenitor cells that is not contacted with the at least one perturbagen.

Embodiment 56. The method of Embodiment 43, wherein the ratio of the number of goblet cells, Paneth cells, and enteroendocrine cells to the number of enterocytes is increased relative to the ratio in the population of progenitor cells prior to contacting with the at least one perturbagen.

Embodiment 57. The method of any of Embodiments 1 to 32, wherein the number of goblet progenitors, goblet cells, Paneth cells, enterocyte progenitor cells, and/or enterocytes is decreased.

Embodiment 58. The method of any of Embodiments 1 to 32, wherein the number of goblet progenitors is decreased.

Embodiment 59. The method of any of Embodiments 1 to 32, wherein the number of goblet cells is decreased.

Embodiment 60. The method of any of Embodiments 1 to 32, wherein the number of Paneth cells is decreased.

Embodiment 61. The method of any of Embodiments 1 to 32, wherein the number of enterocyte progenitor cells is decreased.

Embodiment 62. The method of any of Embodiments 1 to 32, wherein the number of enterocytes is decreased.

Embodiment 63. The method of any of Embodiments 1 to 32, wherein the number of goblet progenitors, goblet cells, Paneth cells, and/or enteroendocrine cells is increased.

Embodiment 64. The method of any of Embodiments 1 to 32, wherein the number of goblet progenitors is increased.

Embodiment 65. The method of any of Embodiments 1 to 32, wherein the number of goblet cells is increased.

Embodiment 66. The method of any of Embodiments 1 to 32, wherein the number of Paneth cells is increased.

Embodiment 67. The method of any of Embodiments 1 to 32, wherein the number of enteroendocrine cells is increased.

Embodiment 68. The method of any one of Embodiments 1 to 67, wherein the at least one perturbagen selected from Table 2, or a variant thereof, comprises at least 2, at least 3, at least 4, at least 5, at least 6, at least 7, at least 8, at least 9, at least 10, at least 11, or all 12 perturbagens selected from Table 2, or variants thereof.

Embodiment 69. The method of any one of Embodiments 1 to 67, wherein the one or more genes are selected from the genes designated as an “up” gene in the gene directionality column of Table 1 comprises 2 or more, 3 or more, 4 or more, 5 or more, 6 or more, or 7 genes selected from the genes designated as an “up” gene in the gene directionality column of Table 1.

Embodiment 70. The method of Embodiment 69, wherein the one or more genes selected from Table 1 comprises at least one of NPDC1, SOX4, BAMBI, DRAP1, SLC25A4, CDK4, and SMARCD2.

Embodiment 71. The method of any one of Embodiments 1 to 67, wherein the one or more genes are selected from the genes designated as a “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, or 68 genes selected from the genes designated as a “down” gene in the gene directionality column of Table 1.

Embodiment 72. The method of Embodiment 71, wherein the one or more genes selected from Table 1 comprises at least one of CD44, DCTD, CDK6, GAA, DDB2, HMGA2, ST7, SLC35F2, MLEC, DPH2, MBNL1, JADE2, MIF, SLC5A6, C2CD2, CRTAP, ATF1, PPIE, ADCK3, HES1, ATP1B1, TIMM9, MYC, MAP3K4, CHERP, TBP, DAG1, TEX10, BAG3, NET1, FZD7, RAD9A, NUDT9, PIK3R4, MRPL12, FPGS, ANXA7, HN1L, METRN, LYN, TGFBR2, STAT5B, RAC2, MALT1, DHX29, EPHB2, CDC25B, PIH1D1, GTPBP8, RBM15B, ELOVL6, IKBKAP, SLC25A13, HSPD1, TSEN2, HEATR1, ME2, BACE2, RFX5, BDH1, PPARG, SLC37A4, NNT, DNM1, ICMT, ETFB, NCK2, and CCND1.

Embodiment 73. The method of any one of Embodiments 1 to 72, wherein contacting the population of progenitor cells occurs in vitro or ex vivo.

Embodiment 74. The method of any one of Embodiments 1 to 72, wherein contacting the population of progenitor cells occurs in vivo in a subject.

Embodiment 75. The method of Embodiment 74, wherein the subject is a human.

Embodiment 76. The method of any one of Embodiments 1 to 75, wherein the change in cells state provides increased secretion of cholecystokinin (CCK), glucagon-like peptide 1 and 2 (GLP-1 and GLP-2), glucose dependent insulinotropic peptide (GIP), peptide YY (PYY), gastrin, secretin, somatostatin, motilin, leptin, nesfatin-1, and ghrelin, bioactive amines, histamine, serotonin (5-HT), neurotensin, vasoactive intestinal peptide, and enteroglucagon by an enteroendocrine cell.

Embodiment 77. A perturbagen for use in the method of any one of Embodiments 1 to 76.

Embodiment 78. A pharmaceutical composition comprising the perturbagen of Embodiment 77.

Embodiment 79. A method for promoting the formation of an enteroendocrine cell, or an immediate progenitor thereof, comprising: exposing a starting population of intestinal stem cells to a perturbation having a perturbation signature that promotes the transition of the starting population of intestinal stem cells into a goblet progenitor cell or an enteroendocrine cell, wherein the perturbation signature comprises increased expression and/or activity in the intestinal stem cell of one or more of genes designated as an “up” gene in the gene directionality column of Table 1 and/or a decreased expression and/or activity in the intestinal stem cell of one or more genes designated as a “down” gene in the gene directionality column of Table 1

Embodiment 80. The method of Embodiment 79, wherein the perturbation signature comprises an increase in expression and/or activity of one or more genes in the progenitor cell of an activation of a network module designated in the network module column of Table 1.

Embodiment 81. The method of Embodiment 79 or Embodiment 80, wherein the activation of one or more genes of the network module designated in the network module column of Table 1 comprises modulating expression and/or activity of 2 or more genes within a network module.

Embodiment 82. The method of any one of Embodiments 79-81, wherein the perturbation signature comprises an increase in expression and/or activity in the progenitor cell of two or more genes designated as an “up” gene in the gene directionality column of Table 1.

Embodiment 83. The method of any one of Embodiments 79-82, wherein the perturbation signature comprises a decrease in expression and/or activity in the progenitor cell of two or more genes designated as a “down” gene in the gene directionality column of Table 1.

Embodiment 84. A method for treating a disease or disorder characterized by an abnormal number of enteroendocrine cells, comprising: (a) administering to a patient in need thereof a therapeutically effective amount of at least one perturbagen selected from Table 2, or a variant thereof, wherein the at least one perturbagen is capable of changing a gene signature in a progenitor cell, or (b) administering to a patient in need thereof a cell, the cell having been contacted with at least one perturbagen selected from Table 2, or a variant thereof, wherein the at least one perturbagen is capable of changing a gene signature in a progenitor cell.

Embodiment 85. The method of Embodiment 84, wherein the disease or disorder is caused by an enteroendocrine cell deficiency.

Embodiment 86. The method of Embodiments 84 or 85, wherein the administering is directed to the bone marrow of the patient.

Embodiment 87. The method of Embodiment 86, wherein the administering is via intraosseous injection or intraosseous infusion.

Embodiment 88. The method of any one of Embodiments 84 to 87, wherein the administering the cell is via intravenous injection or intravenous infusion.

Embodiment 89. The method of any one of Embodiments 84 to 88, wherein the administering is simultaneously or sequentially to one or more mobilization agents.

Embodiment 90. The method of Embodiment 84, wherein the administering of the perturbagen is via oral, intravenous, intramuscular, intraperitoneal, subcutaneous, intra-articular injection, and/or infusion route.

Embodiment 91. The method of Embodiment 90, wherein the delivery is via the gastrointestinal (GI) tract, optionally selected from the stomach, small intestine, duodenum, jejunum, ileum, large intestine, colon transversum, colon descendens, colon ascendens, colon sigmoidenum, cecum, and rectum.

Embodiment 92. The method of Embodiments 90 or 91, wherein the perturbagen is formulated with a delayed-release coating, which is optionally enzyme-dependent.

Embodiment 93. The method of Embodiment 84, wherein the disease or disorder is selected from Type II Diabetes, obesity, weight loss, intestinal inflammation (e.g. inflammatory bowel disease, infection, colorectal cancer or food allergies), nonalcoholic fatty liver disease, and cardiovascular complications of diabetes.

Embodiment 94. The method of Embodiment 85, wherein at least one perturbagen is administered on the basis of previously determining that the patient exhibits an abnormal number of enteroendocrine cells, or a disease or disorder characterized thereby.

Embodiment 95. A method for treating a disease or disorder characterized by an abnormal ratio of enteroendocrine cells to intestinal stem cells, comprising: (a) administering to a patient in need thereof at least one perturbagen selected from Table 2, or a variant thereof, wherein the at least one perturbagen is capable of changing a gene signature in an intestinal stem cell or (b) administering to a patient in need thereof a cell, the cell having been contacted with at least one perturbagen selected from Table 2, or a variant thereof, wherein the at least one perturbagen is capable of changing a gene signature in an intestinal stem cell.

Embodiment 96. The method of Embodiment 95, wherein the abnormal ratio comprises a decreased number of enteroendocrine cells and/or an increased number of intestinal stem cells.

Embodiment 97. The method of Embodiment 96, wherein the abnormal ratio comprises an increased number of intestinal stem cells.

Embodiment 98. The method of Embodiment 96, wherein the abnormal ratio comprises a decreased number of enteroendocrine cells.

Embodiment 99. The method of Embodiment 95, wherein the administering is directed to the bone marrow of the patient.

Embodiment 100. The method of Embodiment 95, wherein the administering is via intraosseous injection or intraosseous infusion.

Embodiment 101. The method of any one of Embodiments 95 to 100, wherein the administering the cell is via intravenous injection or intravenous infusion.

Embodiment 102. The method of any one of Embodiments 95 to 101, wherein the administering is simultaneously or sequentially to one or more mobilization agents.

Embodiment 103. The method of Embodiment 95, wherein the administering of the perturbagen is via oral, intravenous, intramuscular, intraperitoneal, subcutaneous, intra-articular injection, and/or infusion route.

Embodiment 104. The method of Embodiment 103, wherein the delivery is via the gastrointestinal (GI) tract, optionally selected from the stomach, small intestine, duodenum, jejunum, ileum, large intestine, colon transversum, colon descendens, colon ascendens, colon sigmoidenum, cecum, and rectum.

Embodiment 105. The method of Embodiment 95, wherein the disease or disorder is selected from Type II Diabetes, obesity, weight loss, intestinal inflammation (e.g. inflammatory bowel disease, infection, colorectal cancer or food allergies), nonalcoholic fatty liver disease, and cardiovascular complications of diabetes.

Embodiment 106. The method of Embodiment 95, wherein at least one perturbagen is administered on the basis of previously determining that the patient exhibits the abnormal ratio of enteroendocrine cells to intestinal stem cells, or a disease or disorder characterized thereby.

Embodiment 107. The method of any one of Embodiments 95 to 106, wherein the at least one perturbagen is capable of changing a gene signature in a progenitor cell.

Embodiment 108. The method of any one of Embodiments 95 to 107, wherein the patient is selected by steps comprising: obtaining from the patient having the disease or disorder a sample of cells comprising at least one intestinal stem cell; and contacting the sample of cells with least one perturbagen selected from Table 2, or a variant thereof, wherein the at least one perturbagen alters a gene signature in the sample of cells.

Embodiment 109. The method of any one of Embodiments 95 to 107, wherein the patient is selected by steps comprising: obtaining from a subject having the disease or disorder a sample of cells comprising at least one intestinal stem cell; and contacting the sample of cells with at least one perturbagen capable of altering a gene signature in an intestinal stem cell, wherein the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1.

Embodiment 110. The method of Embodiment 109, wherein the perturbagen causes an increase in expression and/or activity of one or more genes in the progenitor cell of an activation of a network module designated in the network module column of Table 1.

Embodiment 111. The method of Embodiment 109 or Embodiment 110, wherein the activation of one or more genes of the network module designated in the network module column of Table 1 comprises modulating expression and/or activity of 2 or more genes within a network module.

Embodiment 112. The method of any one of Embodiments 109-111, wherein the perturbagen causes an increase in expression and/or activity in the progenitor cell of two or more genes designated as an “up” gene in the gene directionality column of Table 1.

Embodiment 113. The method of any one of Embodiments 109-112, wherein the perturbagen causes a decrease in expression and/or activity in the progenitor cell of two or more genes designated as a “down” gene in the gene directionality column of Table 1.

Embodiment 114. The method of any one of Embodiments 95 to 107, wherein the patient is selected by steps comprising: obtaining from a subject having the disease or disorder a sample of cells comprising an intestinal stem cell; and contacting the sample of cells with at least one perturbagen selected from Table 2, or a variant thereof; wherein the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1

Embodiment 115. The method of Embodiment 114, wherein the perturbagen causes an increase in expression and/or activity of one or more genes in the progenitor cell of an activation of a network module designated in the network module column of Table 1.

Embodiment 116. The method of Embodiment 114 or Embodiment 115, wherein the activation of one or more genes of the network module designated in the network module column of Table 1 comprises modulating expression and/or activity of 2 or more genes within a network module.

Embodiment 117. The method of any one of Embodiments 114-116, wherein the perturbagen causes an increase in expression and/or activity in the progenitor cell of two or more genes designated as an “up” gene in the gene directionality column of Table 1.

Embodiment 118. The method of any one of Embodiments 114-117, wherein the perturbagen causes a decrease in expression and/or activity in the progenitor cell of two or more genes designated as a “down” gene in the gene directionality column of Table 1.

Embodiment 119. A method for selecting the patient of any one of Embodiments 95 to 107, comprising: obtaining from a subject having the disease or disorder a sample of cells comprising an intestinal stem cell; and contacting the sample of cells with least one perturbagen selected from Table 2, or a variant thereof, wherein when the at least one perturbagen alters a gene signature in the sample of cells, the subject is selected as a patient.

Embodiment 120. A method for selecting the patient of any one of Embodiments 95 to 107, comprising: obtaining from a subject having the disease or disorder a sample of cells comprising an intestinal stem cell; and contacting the sample of cells with at least one perturbagen capable of altering a gene signature in an intestinal stem cell, wherein when the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1, the subject is selected as a patient.

Embodiment 121. The method of Embodiment 120, wherein altering the gene signature comprises a change in expression and/or activity of one or more genes in the progenitor cell of a network module designated in the network module column of Table 1.

Embodiment 122. The method of any one of Embodiment 120 or Embodiment 121, wherein the activation of one or more genes of the network module designated in the network module column of Table 1 comprises modulating expression and/or activity of 2 or more genes within a network module.

Embodiment 123. The method of any one of Embodiments 120-122, wherein altering the gene signature comprises an increase in expression and/or activity in the progenitor cell of two or more genes designated as an “up” gene in the gene directionality column of Table 1.

Embodiment 124. The method of any one of Embodiments 120-123, wherein altering the gene signature comprises a decrease in expression and/or activity in the progenitor cell of two or more genes designated as a “down” gene in the gene directionality column of Table 1.

Embodiment 125. A method for selecting the patient of any one of Embodiments 95 to 107, comprising: obtaining from a subject having the disease or disorder a sample of cells comprising an intestinal stem cell; and contacting the sample of cells with at least one perturbagen selected from Table 2, or a variant thereof; wherein when the at least one perturbagen increases in the sample of cells the expression and/or activity of one or more genes designated as an “up” gene in the gene directionality column of Table 1 and/or decreases in the sample of cells the expression and/or activity of one or more genes designated as a “down” gene in the gene directionality column of Table 1, the subject is selected as a patient.

Embodiment 126. The method of Embodiment 125, wherein the perturbagen causes an increase in expression and/or activity of one or more genes in the progenitor cell of an activation of a network module designated in the network module column of Table 1.

Embodiment 127. The method of Embodiment 125 or Embodiment 126, wherein the activation of one or more genes of the network module designated in the network module column of Table 1 comprises modulating expression and/or activity of 2 or more genes within a network module.

Embodiment 128. The method of any one of Embodiments 125-127, wherein the perturbagen causes an increase in expression and/or activity in the progenitor cell of two or more genes designated as an “up” gene in the gene directionality column of Table 1.

Embodiment 129. The method of any one of Embodiments 125-128, wherein the perturbagen causes a decrease in expression and/or activity in the progenitor cell of two or more genes designated as a “down” gene in the gene directionality column of Table 1.

Embodiment 130. Use of the perturbagen of Table 2, or a variant thereof in the manufacture of a medicament for treating a disease or disorder characterized by an abnormal ratio of enteroendocrine cells to intestinal stem cells.

Embodiment 131. Use of the perturbagen of Table 2, or a variant thereof in the manufacture of a medicament for treating a disease or disorder characterized by an abnormal ratio of enteroendocrine cells to enterocytes, Paneth cells and/or goblet cells.

Embodiment 132. A method of identifying a candidate perturbation for promoting the transition of a starting population of intestinal stem cells into enteroendocrine cells or immediate progenitors thereof, the method comprising: exposing the starting population of intestinal stem cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of intestinal stem cells into enteroendocrine cells or immediate progenitors thereof following exposure of the population of cells to the perturbation; and identifying the perturbation as a candidate perturbation for promoting the transition of a population of intestinal stem cells into enteroendocrine cells or immediate progenitors thereof based on the perturbation signature, wherein the perturbation signature is an increase in expression and/or activity in the intestinal stem cell of one or more genes designated as an “up” gene in the gene directionality column of Table 1, and/or a decrease in expression and/or activity in the intestinal stem cell of one or more genes designated as a “down” gene in the gene directionality column of Table 1.

Embodiment 133. The method of Embodiment 132, wherein the perturbation signature is an increase in expression and/or activity in the progenitor cell of an activation of a network module designated in the network module column of Table 1.

Embodiment 134. The method of Embodiment 132 or Embodiment 133, wherein the activation of one or more genes of the network module designated in the network module column of Table 1 comprises modulating expression and/or activity of 2 or more genes within a network module.

Embodiment 135. The method of any one of Embodiments 132-134, wherein the perturbation signature is an increase in expression and/or activity in the progenitor cell of two or more genes designated as an “up” gene in the gene directionality column of Table 1.

Embodiment 136. The method of any one of Embodiments 132-135, wherein the perturbation signature is a decrease in expression and/or activity in the progenitor cell of two or more genes designated as a “down” gene in the gene directionality column of Table 1.

Embodiment 137. A method for making a therapeutic agent for a disease or disorder selected from Type II Diabetes, obesity, weight loss, intestinal inflammation (e.g. inflammatory bowel disease, infection, colorectal cancer or food allergies), nonalcoholic fatty liver disease, and cardiovascular complications of diabetes, comprising: (a) identifying a candidate perturbation according to the method of Embodiment 132, and (b) formulating the candidate perturbation as a therapeutic agent for the treatment of the disease or disorder.

Embodiment 138. A method for directing a change in cell state of a progenitor cell, comprising: contacting a population of cells comprising a progenitor cell with at least one perturbagen capable of altering a gene signature in the progenitor cell; wherein altering the gene signature comprises an increase in expression and/or activity in the progenitor cell of one or more genes designated as an “up” gene in the gene directionality column of Table 1 and/or a decrease in expression and/or activity in the progenitor cell of one or more genes designated as a “down” gene in the gene directionality column of Table 1; wherein the progenitor cell is an intestinal stem cell and wherein the change in cell state provides an increase in the number of one or more of enteroendocrine cells, goblet progenitors, goblet cells, and Paneth cells.

Embodiment 139. The method of Embodiment 138, wherein the change in cell state provides an increase in the number of enteroendocrine cells.

Embodiment 140. The method of Embodiment 138, wherein the increase in the number of enteroendocrine cells is relative to the number of enteroendocrine cells obtained from a population of progenitor cells that is not contacted with the at least one perturbagen and/or relative to the number of enteroendocrine cells obtained from a population of progenitor cells prior to contacting with the at least one perturbagen.

Embodiment 141. The method of Embodiment 138, wherein the at least one perturbagen is selected from Table 2, or a variant thereof. In embodiments, the at least one perturbagen selected from Table 2, or a variant thereof, comprises at least 2, at least 3, at least 4, at least 5, at least 6, at least 7, at least 8, at least 9, at least 10, at least 11, or all 12 perturbagens selected from Table 2, or variants thereof.

Embodiment 142. The method of Embodiment 138, wherein the one or more genes selected from Table 1 comprises at least one of CD44, DCTD, CDK6, GAA, DDB2, HMGA2, ST7, SLC35F2, MLEC, DPH2, MBNL1, JADE2, MIF, SLC5A6, C2CD2, CRTAP, ATF1, PPIE, ADCK3, HES1, ATP1B1, TIMM9, MYC, MAP3K4, CHERP, TBP, DAG1, TEX10, BAG3, NET1, FZD7, RAD9A, NUDT9, PIK3R4, MRPL12, FPGS, ANXA7, HN1L, METRN, LYN, TGFBR2, STAT5B, RAC2, MALT1, DHX29, EPHB2, CDC25B, PIH1D1, GTPBP8, RBM15B, ELOVL6, IKBKAP, SLC25A13, HSPD1, TSEN2, HEATR1, ME2, BACE2, RFX5, BDH1, PPARG, SLC37A4, NNT, DNM1, ICMT, ETFB, NCK2, and CCND1.

Methods of Culturing Cells In Vitro to Perform Single-Cell Analyses

In carrying out the techniques described herein for identifying the causes of cell fate, it is useful to generate datasets regarding cellular-component measurements obtained from single-cells. To generate these datasets, a population of cells of interest may be cultured in vitro. Alternately, these datasets may be generated, from single cells that have not been previously cultured; for example, cells used in single cell analyses may be obtained from dissociated primary tissue or from a blood product. This latter method of generating datasets is often desirable if one wants to capture information of the primary cell/organ as close to the in vivo setting as possible. However, for cells undergoing culturing, single-cell measurements of one or more cellular-components of interest may be performed at one or more time periods during the culturing to generate datasets.

In some embodiments, cellular-components of interest include nucleic acids, including DNA, modified (e.g., methylated) DNA, RNA, including coding (e.g., mRNAs) or non-coding RNA (e.g., sncRNAs), proteins, including post-transcriptionally modified protein (e.g., phosphorylated, glycosylated, myristilated, etc. proteins), lipids, carbohydrates, nucleotides (e.g., adenosine triphosphate (ATP), adenosine diphosphate (ADP) and adenosine monophosphate (AMP)) including cyclic nucleotides such as cyclic adenosine monophosphate (cAMP) and cyclic guanosine monophosphate (cGMP), other small molecule cellular-components such as oxidized and reduced forms of nicotinamide adenine dinucleotide (NADP/NADPH), and any combinations thereof. In some embodiments, the cellular-component measurements comprise gene expression measurements, such as RNA levels.

Any one of a number of single-cell cellular-component expression measurement techniques may be used to collect the datasets. Examples include, but are not limited to single-cell ribonucleic acid (RNA) sequencing (scRNA-seq), scTag-seq, single-cell assay for transposase-accessible chromatin using sequencing (scATAC-seq), CyTOF/SCoP, E-MS/Abseq, miRNA-seq, CITE-seq, and so on. The cellular-component expression measurement can be selected based on the desired cellular-component to be measured. For instance, scRNA-seq, scTag-seq, and miRNA-seq measure RNA expression. Specifically, scRNA-seq measures expression of RNA transcripts, scTag-seq allows detection of rare mRNA species, and miRNA-seq measures expression of micro-RNAs. CyTOF/SCoP and E-MS/Abseq measure protein expression in the cell. CITE-seq simultaneously measures both gene expression and protein expression in the cell. And scATAC-seq measures chromatin conformation in the cell. Table 3 below provides links to example protocols for performing each of the single-cell cellular-component expression measurement techniques described herein.

TABLE 3
Example Measurement Protocols

| Technique | Protocol |
|---|---|
| RNA-seq | Olsen and Baryawno “Introduction to Single-Cell RNA Sequencing” |
| | Current Protocols in Molecular Biology. Volume 122, Issue 1, April 2018, |
| | e57 |
| Tag-seq | Rozenberg et al., “Digital gene expression analysis with sample |
| | multiplexing and PCR duplicate detection: A straightforward protocol”, |
| | BioTechniques, vol. 61, No. 1, March 2018 |
| ATAC-seq | Buenrostro et al., “ATAC-seq: A Method for Assaying Chromatin |
| | Accessibility Genome-Wide”, Curr Protoc Mol Biol. 2015; 109: 21.29.1- |
| | 21.29.9 |
| miRNA-seq | Faridani et al., “Single-cell sequencing of the small-RNA transcriptome” |
| | Nature Biotechnology volume 34, pages 1264-1266 (2016) |
| CyTOF/SCoPE- | Bandura et al., “Mass Cytometry: Technique for Real Time Single Cell |
| MS/Abseq | Multitarget Immunoassay Based on Inductively Coupled Plasma Time-of- |
| | Flight Mass Spectrometry”, Anal Chem. 2009 Aug 15; 81(16): 6813-22 |
| | Shahi et al., “Abseq: Ultrahigh-throughput single cell protein profiling with |
| | droplet microfluidic barcoding”, Scientific Reports volume 7, Article |
| | number: 44447 (2017) |
| | Budnik et al., “SCoPE-MS: mass spectrometry of single mammalian cells |
| | quantifies proteome heterogeneity during cell differentiation”, Genome |
| | Biology 201819: 161 |
| CITE-seq | Stoeckius et al., “Simultaneous epitope and transcriptome measurement |
| | in single cells”, Nature Methods, vol 14, pages 865-868 (2017) |

The cellular-component expression measurement technique used may result in cell death. Alternatively, cellular-components may be measured by extracting out of the live cell, for example by extracting cell cytoplasm without killing the cell. Techniques of this variety allow the same cell to be measured at multiple different points in time.

If the cell population is heterogeneous such that multiple different cell types that originate from a same “progenitor” cell are present in the population, then single-cell cellular-component expression measurements can be performed at a single time point or at relatively few time points as the cells grow in culture. As a result of the heterogeneity of the cell population, the collected datasets will represent cells of various types along a trajectory of transition.

If the cell population is substantially homogeneous such that only a single or relatively few cell types, mostly the “progenitor” cell of interest, are present in the population, then single-cell cellular-component expression measurements can be performed multiple times over a period of time as the cells transition.

A separate single-cell cellular-component expression dataset is generated for each cell, and where applicable at each of the time periods. The collection of single-cell cellular-component expression measurements from a population of cells at multiple different points in time can collectively be interpreted as a “pseudo-time” representation of cell expression over time for the cell types originating from the same “progenitor” cell. The term pseudo-time is used in two respects, first, in that cell state transition is not necessarily the same from cell to cell, and thus the population of cell provides a distribution of what transition processes a cell of that “progenitor” type is likely to go through over time, and second, that the cellular-component expression measurements of those multiple cell's expressions at multiple time points simulates the possible transition behavior over time, even if cellular-component expression measurements of distinct cells give rise to the datasets. As a deliberately simple example, even if cell X gave a dataset for time point A and cell Y gave a dataset for time point B, together these two datasets represent the pseudo-time of transition between time point A and time point B.

For convenience of description, two such datasets captured for a “same” cell at two different time periods (assuming a technique is used that does not kill the cell) are herein referred to as different “cells” (and corresponding different datasets) because in practice such cells will often be slightly or significantly transitioned from each other, in some cases having an entirely distinct cell type as determined from the relative quantities of various cellular-components. Viewed from this context, these two measurements of a single-cell at different time points can be interpreted as different cells for the purpose of analysis because the cell itself has changed.

Note that the separation of datasets by cell/time period described herein is for clarity of description, in practice, these datasets may be stored in computer memory and logically operated on as one or more aggregate dataset/s (e.g., by cell for all time periods, for all cells and time periods at once).

In some instances, it is useful to collect datasets where a “progenitor” cell of interest has been perturbed from its base line state. There are a number of possible reasons to do this, for example, to knock out one or more cellular-components, to evaluate the difference between healthy and diseased cell states. In these instances, a process may also include steps for introducing the desired modifications to the cells. For example, one or more perturbations may be introduced to the cells, tailored viruses designed to knock out one or more cellular-components may be introduced, clustered regularly interspaced short palindromic repeats (CRISPR) may be used to edit cellular-components, and so on. Examples of techniques that could be used include, but are not limited to, RNA interference (RNAi), Transcription activator-like effector nuclease (TALEN) or Zinc Finger Nuclease (ZFN).

Depending upon how the perturbation is applied, not all cells will be perturbed in the same way. For example, if a virus is introduced to knockout a particular gene, that virus may not affect all cells in the population. More generally, this property can be used advantageously to evaluate the effect of many different perturbations with respect to a single population. For example, a large number of tailored viruses may be introduced, each of which performs a different perturbation such as causing a different gene to be knocked out. The viruses will variously infect some subset of the various cells, knocking out the gene of interest. Single-cell sequencing or another technique can then be used to identify which viruses affected which cells. The resulting differing single-cell sequencing datasets can then be evaluated to identify the effect of gene knockout on gene expression in accordance with the methods described elsewhere in this description.

Other types of multi-perturbation cell modifications can be performed similarly, such as the introduction of multiple different perturbations, barcoding CRISPR, etc. Further, more than one type perturbation may be introduced into a population of cells to be analyzed. For example, cells may be affected differently (e.g., different viruses introduced), and different perturbations may be introduced into different sub-populations of cells.

Additionally, different subsets of the population of cells may be perturbed in different ways beyond simply mixing many perturbations and post-hoc evaluating which cells were affected by which perturbations. For example, if the population of cells is physically divided into different wells of a multi-well plate, then different perturbations may be applied to each well. Other ways of accomplishing different perturbations for different cells are also possible.

Below, methods are exemplified using single-cell gene expression measurements. It is to be understood that this is by way of illustration and not limitation, as the present disclosure encompasses analogous methods using measurements of other cellular-components obtained from single-cells. It is to be further understood that the present disclosure encompasses methods using measurements obtained directly from experimental work carried out by an individual or organization practicing the methods described in this disclosure, as well as methods using measurements obtained indirectly, e.g., from reports of results of experimental work carried out by others and made available through any means or mechanism, including data reported in third-party publications, databases, assays carried out by contractors, or other sources of suitable input data useful for practicing the disclosed methods.

As discussed herein, gene expression in a cell can be measured by sequencing the cell and then counting the quantity of each gene transcript identified during the sequencing. In some embodiments, the gene transcripts sequenced and quantified may comprise RNA, for example mRNA. In alternative embodiments, the gene transcripts sequenced and quantified may comprise a downstream product of mRNA, for example a protein such as a transcription factor. In general, as used herein, the term “gene transcript” may be used to denote any downstream product of gene transcription or translation, including post-translational modification, and “gene expression” may be used to refer generally to any measure of gene transcripts.

Although the remainder of this description focuses on the analysis of gene transcripts and gene expression, all of the techniques described herein are equally applicable to any technique that obtains data on a single-cell basis regarding those cells. Examples include single-cell proteomics (protein expression), chromatin conformation (chromatin status), methylation, or other quantifiable epigenetic effects.

The following description provides an example general description for culturing a population of cells in vitro in order to carry out single-cell cellular-component expression measurement multiple time periods. Methods for culturing cells in vitro are known in the art. Those of skill in the art will also appreciate how this process could be modified for longer/shorter periods, for additional/fewer single-cell measurement steps, and so on.

In one embodiment, the process for culturing cells in a first cell state into cells in a second cell state includes one or more of the following steps:
Day 0: Thaw cells in the first cell state into a plate in a media suitable for growth of the cells.
Day 1: Seed cells in the first cell state into a multi-well plate. If applicable, perform additional steps to affect gene expression by cells. For example, simultaneously infect with one or more viruses to activate or knock out genes of interest.
   Perform gene expression measurement iteration t₁ for cells in the wells.
Day 1+l: Change media as needed if any additional processes are performed.
   If applicable, perform gene expression measurement iteration tₗ for cells in the wells.
Day 1+m: Change media to media appropriate to support growth of cells in the second cell state.
   If applicable, perform gene expression measurement iteration tₘ for cells in the wells.
Days 1+n, o, p, etc.: Media change as needed to support further cell state transition from the first cell state to the second cell state. If applicable, perform additional steps to affect further transition from the first cell state to the second cell state. For example, add perturbations of interest to push cells towards the second cell state.
   If applicable, perform gene expression measurement iterations tₙ, tₒ, tₚ, etc., for cells in the wells.
Day q: Perform gene expression measurement iteration t_{q} for cells in the wells and in the second state.
Collect cells into a tube and stain in suspension with antibodies matched to genes/proteins of interest, thereby sorting/identifying cells without having to lyse/destroy them. This step also can identify surface proteins that might not be seen with as much resolution in the setting of the cytoplasm. Image with a cell imaging system such as the BD Celestra flow cytometer or similar instrument by acquiring the cells from each well or tube. Quantify of number of cells per well that are in the first cell state and the number of cells per well that are in the second cell state. These steps can be used with unfixed cells.

Any aspect or embodiment disclosed herein can be combined with any other aspect or embodiment as disclosed herein.

### Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

Any terms not directly defined herein shall be understood to have the meanings commonly associated with them as understood within the art of the disclosure. Certain terms are discussed herein to provide additional guidance to the practitioner in describing the compositions, the devices, the methods and the like of aspects of the disclosure and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the disclosure herein.

The term “perturbation” in reference to a cell (e.g., a perturbation of a cell or a cellular perturbation) refers to any treatment of the cell with one or more active agents capable of causing a change in the cell's lineage or cell state (or in the lineage or cell state of the cell's progeny). These active agents can be referred to as “perturbagens.” In embodiments, the perturbagen can comprise, e.g., a small molecule, a biologic, a protein, a protein combined with a small molecule, an antibody-drug conjugate (ADC), a nucleic acid, such as an siRNA or interfering RNA, a cDNA over-expressing wild-type and/or mutant shRNA, a cDNA over-expressing wild-type and/or mutant guide RNA (e.g., Cas9 system, Cas9-gRNA complex, or other gene editing system), or any combination of any of the foregoing. As used herein, a perturbagen classified as a “compound” may be a small molecule or a biologic. Also, a perturbagen classified as “overexpression of gene” may be cDNA over-expressing a wild-type gene or an mRNA encoding a wild-type gene. In embodiments, an mRNA may comprise a modified nucleotide that promotes stability of the mRNA and/or reduces toxicity to a subject. Examples of modified nucleotides useful in the present disclosure include pseudouridine and 5-methylcytidine. Where a perturbagen is (or includes) a nucleic acid or protein described by reference to a particular sequence, it should be understood that variants with similar function and nucleic acid or amino acid identity are encompassed as well, e.g., variants with about: 1%, 2%, 3%, 4%, 5%, 6%, 7%, 8%, 9%, 10%, 11%, 12%, 13%, 14%, 15%, or more, variation, i.e., having about: 99%, 98%, 97%, 96%, 95%, 94%, 93%, 92%, 91%, 90%, 89%, 88%, 87%, 86%, or 85% identity to the reference sequence; e.g., in some embodiments, having, for example, at least: 1, 2, 3, 4, 5, 6, 7, 8, 9, 10, 11, 12, 13, 14, 15, 20, 25, 30, 35, 40, 45, 50, or more, substitutions.

The term “progenitor” in reference to a cell (e.g., a progenitor cell) refers to any cell that is capable of transitioning from one cell state to at least one other cell state. Thus, a progenitor can differentiate into one or more cell types and/or can expand into one or more types of cell populations. In some instances, the term progenitor refers to intestinal stem cells.

As used herein, the terms “cell fate” and “cell state” are interchangeable and synonymous.

The term “subject,” refers to an individual organism such as a human or an animal. In embodiments, the subject is a mammal (e.g., a human, a non-human primate, or a non-human mammal), a vertebrate, a laboratory animal, a domesticated animal, an agricultural animal, or a companion animal. In embodiments, the subject is a human (e.g., a human patient). In embodiments, the subject is a rodent, a mouse, a rat, a hamster, a rabbit, a dog, a cat, a cow, a goat, a sheep, or a pig.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”. Likewise, the term “and/or” covers both “or” and “and”.

Unless specifically stated or obvious from context, as used herein, the term “about” is understood as within a range of normal tolerance in the art, for example within 2 standard deviations of the mean. About is understood to be within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The disclosure will be further described in the following examples, which do not limit the scope of the disclosure described in the claims.

### EXAMPLES

### Example 1: Single Cell Gene Expression Profiling of the Murine Small Intestine and Colon

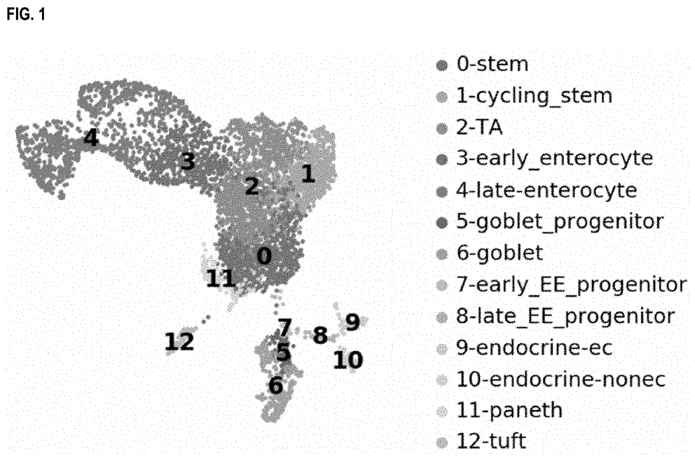

To characterize the transitions that occur during intestinal differentiation, maintenance and disease in order to generate testable hypotheses to prevent disease or restore health, we performed initial analyses and modeling of the in vivo intestinal differentiation based on the publicly available data described in Haber, Adam L., et al. “A single-cell survey of the small intestinal epithelium.” Nature 551.7680 (2017): 333. (FIG. 1). From this analysis, we identified compounds that are predicted to increase enteroendocrine cells within the intestine (TABLE 2), potentially impacting diseases including Type II Diabetes and obesity. These predicted compounds will be tested in vitro and in vivo as described below.

We will perform additional characterization of the murine intestine through single cell RNA sequencing and ATAC sequencing. We will isolate small intestine and colon from C57Bl6/J adult mice (fed chow or high fat diet to induce obesity) and prepare single cell suspensions of intestinal epithelium and lamina propria. The method we will follow is based on the one described in in Smilie et al (2019) Cell. 2019 Jul. 25; 178(3):714-730.e22 paper. Briefly, we will isolate the intestinal epithelium by shaking the tissue in the presence of 20 mM EDTA and we will generate single cell suspensions by enzymatic digestion (TrypLE) followed by mild trituration and filtering through 40 μm cell strainer. The lamina propria will be enzymatically digested and single cells suspensions will be generated by trituration and filtering. The viability of the cells will be assessed by Trypan blue staining and confirmed by flow cytometry using DAPI or PI stain. If the viability is below 90%, we will enrich for live cells by removing dead cells from the cell suspension either by using a dead cell removal kit (i.e. EasySep by Stem Cell Technologies) or FACS sorting. RNA from single cells will be sequenced using inDrop or 10× technology and the data will be curated and further analyzed.

### Example 2: Increasing Enteroendocrine Cell Number In Vitro Using Mouse Small Intestinal Organoids

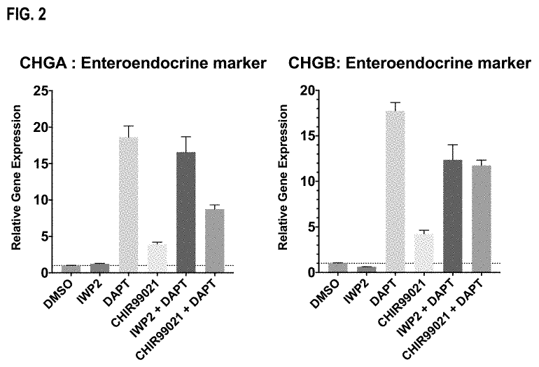

Cryopreserved mouse intestinal organoids derived from small intestine were obtained from Stem Cell Technologies. The organoids were thawed and propagated in Intesticult Growth Medium (Mouse) (purchased from Stem Cell Technologies) following the company's instructions. Briefly, ˜100 organoid fragments/well are embedded in Matrigel domes in 24-well plates and growth media is renewed every 2-3 days. The organoids are passaged every 5-6 days in 1:4 ratio. To assess the potential of the predicted perturbations to induce the enteroendocrine cell lineage during organoid differentiation, we established an in vitro assay to induce the various intestinal lineages (enterocytes, goblet cells, Paneth cells, enteroendocrine cells) based on published data (Yin et al, Nat Methods. 2014 January; 11(1):106-12). Briefly, organoid fragments are embedded in Matrigel and grow in custom-made media in the presence of EGF, Noggin, Rspondin (ENR) supplemented with the indicated factors (Table 4) that are known to induce markers of the various intestinal lineages. The expression of markers and the induction of the various cell lineages was assessed by Q-PCRs after 5 days of treatments (FIG. 2). Further characterization of the organoids will be performed by immunofluorescence staining and flow cytometry. In addition, we will assess the function of the enteroendocrine cells by measuring the amounts of hormones released in the media using ELISA, including GLP-1 and secretin, after stimulation with glucose, glutamine, and short chain fatty acids.

TABLE 4

| | | Markers |
|---|---|---|
| Factors | Cell lineage | (Q-PCRs) |
| IWP2 | Enterocytes, Enteroendocrine Cells | ALPI, ChrA |
| DAPT | Enteroendocrine Cells, Paneth | ChrA, Lyz1 |
| IWP2 + DAPT | Goblet, Enteroendocrine Cells | Muc2, ChrA |
| CHIR99021 + DAPT | Enteroendocrine Cells, Paneth | ChrA, Lyz1 |
| CHIR99021 + | Stem Cells | LGR5 |
| Valproic acid | | |

Following a similar experimental scheme, we will treat intestinal organoids with predicted compounds (Table 2) at various concentrations and we will assess their effects on the induction of enteroendocrine cell lineage as described above.

### Example 3: Increasing the Number of Enteroendocrine Cell Number In Vitro Using Human Small Intestine and Colon Organoids

Cryopreserved human colon organoids from healthy donors or patients with metabolic diseases, including diabetes and obesity, will be obtained from Hubrecht Organoid Technology (HUB). We will use at least 3 independent donors/group. We will use published protocols to culture and differentiate the human organoids in the presence of compounds predicted to increase the number of enteroendocrine cells. Similarly to mouse organoids, we will assess the number and function of enteroendocrine cells by gene expression analyses, imaging, and ELISA techniques in 3D cultures.

### Example 4: Increasing the Number of Enteroendocrine Cell Number In Vivo

We assessed the effects of selected compounds on intestinal cell lineages as a pilot experiment. Following the results described below, we will expand our studies with additional perturbations as described in Example 1 and Table 2. To test the compounds in vivo, adult male BALB/c mice were randomly allocated to experimental groups and allowed to acclimatize for one week. Treatments were administered according to the schedule below (Table 5). From Day 0 until the end of the experiment, animals were weighed and monitored daily for non-specific clinical signs including piloerection, hunched posture and reduced activity. At the end of the experiment, on Day 6, animals were culled, and the colon dissected out. Tissue samples were processed to isolate epithelial cells. One aliquot of cells was fixed and analyzed by flow cytometry using eight (8) markers. Briefly for flow cytometry, colons and small intestines from naïve Balb/c male mice were dissected out, cleaned and processed for flow cytometric analysis of the epithelial fraction using EDTA to remove the epithelium and a brief collagenase digest to dissociate any clumps of cells. Samples were stained for viability and the following panel of markers—CD45, CD31, Ter119, EpCAM, CD117, CD24, DCLK1 and CLCA1 (Tables 6 and 7).

TABLE 5
Treatments were administered according to
the schedule below. All Groups are n = 5

| Group | Dose | Route | Regimen | Necropsy |
|---|---|---|---|---|
| 5% DMSO/Water | 10 mg/kg | IC | SID: Day 0-End | Day 6 |
| Perturbagen 1 | 10 mg/kg | IC | SID: Day 0-End | |
| Perturbagen 2 | 10 mg/kg | IC | SID: Day 0-End | |
| Perturbagen 3 | 4.25 mg/kg | IC | SID: Day 0-End | |
| Perturbagen 4 | 10 mg/kg | IC | SID: Day 0-End | |
| Perturbagen 5 | 10 mg/kg | IC | SID: Day 0-End | |
| Perturbagen 6 | 10 mg/kg | IC | SID: Day 0-End | |

IC = intracolonic delivery;
SID = single injection daily

TABLE 6
Antibodies and conjugated fluorophore for intestinal
cell identification by flow cytometry

| Antibody | Fluorophore | Vendor | Clone |
|---|---|---|---|
| CD45 | PE | eBioscience | 30-F11 |
| CD31 | PE | Biolegend | Mec13.3 |
| Ter119 | PE | Biolegend | Ter119 |
| EpCAM | PerCPCy5.5 | eBioscience | G8.8 |
| CD117 | APC/Cy7 | Biolegend | 2BS |
| CD24 | BV421 | Biolegend | M1/69 |
| DCLK1 | AF488 | Abcam | EPR6085 |
| CLCA1 | AF647 | Abcam | EPR12554-88 |
| fixable viability | BV510 | | |
| dye 510 | | | |

TABLE 7
Gating strategy for Intestinal cell characterization

| | | Expected | |
|---|---|---|---|
| | | frequency | Other |
| Population | Gating Strategy * | (colon) | markers |
| Stem/TA | CD24^{lo}cKit⁻SSC^{lo} | | Lgr5 |
| Enterocytes | | 80% | Vil1 |
| Enteroendocrine | CD24^{hi} cKit⁻ | 1% | CHG-A |
| Goblet | CD24⁺cKit⁺CLCA1⁺ | 16% | Muc2 |
| Tuft | CD24⁺cKit⁺CLCA1⁻DCLK1⁺ | | |
| Paneth | CD24^{hi} cKit^{hi} | Not present/ | |
| | | v. low | |

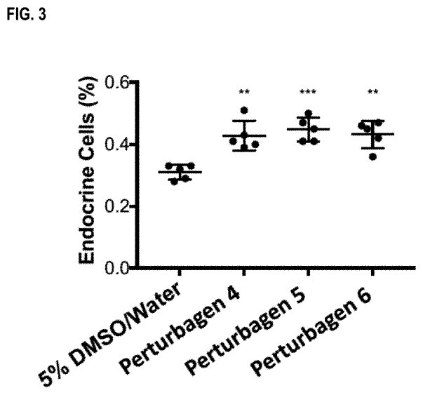

Flow analysis revealed that mice treated with Perturbagen 2 and Perturbagen 6, 2 of 2 (100%) compounds predicted to increase the number of enteroendocrine cells, showed a significant increase in the frequency of enteroendocrine cells within the colonic epithelium (FIG. 3). Perturbagen 5, another compound tested but not predicted to increase the enteroendocrine lineage (1 of 4, 25%) also significantly increased the frequency of enteroendocrine but the other three compounds (Perturbagen 1, Perturbagen 2 and Perturbagen 3) showed no changes in enteroendocrine frequency. Perturbagen 4, Perturbagen 6 and Perturbagen 5 Iso increase the total numbers of epithelial cells within the colon (data not shown) resulting in increased number of enteroendocrine cells.

We predicted compounds that were able to increase the number and frequency of enteroendocrine cells in vivo.

### Example 5: Increasing Enteroendocrine Cells to Treat Obesity and Diabetes In Vivo

Enteroendocrine cells of the intestine sense gut luminal factors, including nutrients and microbial components, and trigger the secretion of peptide hormones and regulate food intake, digestion, or glucose metabolism. Compounds that are predicted to induce the enteroendocrine lineage (Example 2) will be administered to WT C57Bl6/J mice fed chow or high fat diet for 4-20 weeks. Body weight and metabolic parameters (food intake, glucose, insulin, free fatty acids, cholesterol etc.) will be measured in a weekly basis. Glucose handling will be assessed by glucose tolerance test every 3-4 weeks. GLP-1 in the blood will be measured by ELISA, after overnight fasting and challenge with 2 g/kg glucose. A cohort of mice treated with compounds will be sacrificed and the small intestine and colon will be dissected out, cleaned and a portion will be fixed and used for histological analyses, a portion will be used for RNA analyzes of marker of the intestinal cell lineages and another portion will be analyzed with flow cytometry, using the experimental outline described above.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior disclosure.

As used herein, all headings are simply for organization and are not intended to limit the disclosure in any manner. The content of any individual section may be equally applicable to all sections.

### EQUIVALENTS

While the disclosure has been disclosed in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1.-2. (canceled)

3. A method for directing a change in cell state of a progenitor cell, comprising:
contacting a population of cells comprising a progenitor cell with at least one perturbagen selected from Table 2, or a variant thereof, and capable of altering a gene signature in the progenitor cell;
wherein altering the gene signature comprises an increase in expression and/or activity in the progenitor cell of one or more genes designated as an “up” gene in the gene directionality column of Table 1 and/or a decrease in expression and/or activity in the progenitor cell of one or more genes designated as a “down” gene in the gene directionality column of Table 1; and
wherein the progenitor cell is an intestinal stem cell.

4. The method of claim 3, wherein altering the gene signature comprises a change in expression and/or activity of one or more genes in the progenitor cell of a network module designated in the network module column of Table 1.

5. The method of claim 3, wherein the change in cell state provides an increase or decrease in the number of one or more of enteroendocrine cells, goblet progenitors, goblet cells, and Paneth cells.

6. The method of claim 5, wherein the change in cell state provides an increase in the number of enteroendocrine cells.

7. The method of claim 6, wherein the increase in the number of enteroendocrine cells is relative to the number of enteroendocrine cells obtained from a population of progenitor cells that is not contacted with the at least one perturbagen and/or relative to the number of enteroendocrine cells obtained from a population of progenitor cells prior to contacting with the at least one perturbagen.

8. The method of claim 3, wherein the number of progenitor cells is decreased.

9. The method of claim 8, wherein the decrease in the number of progenitor cells is relative to the number of progenitor cells in a population of progenitor cells that is not contacted with the at least one perturbagen and/or relative to the number of progenitor cells in the population prior to contacting with the at least one perturbagen.

10. The method of claim 3, wherein the number of progenitor cells is increased.

11. The method of claim 10, wherein the increase in the number of progenitor cells is relative to the number of progenitor cells in a population of progenitor cells that is not contacted with the at least one perturbagen and/or relative to the number of progenitor cells in the population prior to contacting with the at least one perturbagen.

12. The method of claim 5, wherein the number of goblet progenitors, goblet cells, Paneth cells, and/or enteroendocrine cells is increased after contacting the population of cells comprising a progenitor cell with the at least one perturbagen.

13. The method of claim 5, wherein the number of goblet progenitors, goblet cells, Paneth cells, enterocyte progenitor cells, and/or enterocytes is decreased.

14. The method of claim 5, wherein the number of goblet progenitors, goblet cells, Paneth cells, and/or enteroendocrine cells is increased.

15. The method of claim 3, wherein the at least one perturbagen selected from Table 2, or a variant thereof, comprises at least 2, at least 3, at least 4, at least 5, at least 6, at least 7, at least 8, at least 9, at least 10, at least 11, or all 12 perturbagens selected from Table 2, or variants thereof.

16. The method of claim 3, wherein the one or more genes are selected from the genes designated as a “down” gene in the gene directionality column of Table 1 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, or 68 genes selected from the genes designated as a “down” gene in the gene directionality column of Table 1.

17. The method of claim 16, wherein the one or more genes selected from Table 1 comprises at least one of CD44, DCTD, CDK6, GAA, DDB2, HMGA2, ST7, SLC35F2, MLEC, DPH2, MBNL1, JADE2, MIF, SLC5A6, C2CD2, CRTAP, ATF1, PPIE, ADCK3, HES1, ATP1B1, TIMM9, MYC, MAP3K4, CHERP, TBP, DAG1, TEX10, BAG3, NET1, FZD7, RAD9A, NUDT9, PIK3R4, MRPL12, FPGS, ANXA7, HN1L, METRN, LYN, TGFBR2, STAT5B, RAC2, MALT1, DHX29, EPHB2, CDC25B, PIH1D1, GTPBP8, RBM15B, ELOVL6, IKBKAP, SLC25A13, HSPD1, TSEN2, HEATR1, ME2, BACE2, RFX5, BDH1, PPARG, SLC37A4, NNT, DNM1, ICMT, ETFB, NCK2, and CCND1.

18. The method of claim 3, wherein contacting the population of progenitor cells occurs in vitro or ex vivo or in vivo in a subject.

19. (canceled)

20. (canceled)

21. A method for promoting the formation of an enteroendocrine cell, or an immediate progenitor thereof, comprising:
exposing a starting population of intestinal stem cells to a perturbation having a perturbation signature that promotes the transition of the starting population of intestinal stem cells into a goblet progenitor cell or an enteroendocrine cell,
wherein the perturbation signature comprises increased expression and/or activity in the intestinal stem cell of one or more of genes designated as an “up” gene in the gene directionality column of Table 1 and/or a decreased expression and/or activity in the intestinal stem cell of one or more genes designated as a “down” gene in the gene directionality column of Table 1.

22. The method of claim 21, wherein the perturbation signature comprises an increase in expression and/or activity of one or more genes in the progenitor cell of an activation of a network module designated in the network module column of Table 1.

23. A method for treating a disease or disorder characterized by an abnormal number of enteroendocrine cells, comprising:
(a) administering to a patient in need thereof a therapeutically effective amount of at least one perturbagen selected from Table 2, or a variant thereof, wherein the at least one perturbagen is capable of changing a gene signature in a progenitor cell, or
(b) administering to a patient in need thereof a cell, the cell having been contacted with at least one perturbagen selected from Table 2, or a variant thereof, wherein the at least one perturbagen is capable of changing a gene signature in a progenitor cell.

24. The method of claim 23, wherein the disease or disorder is caused by an enteroendocrine cell deficiency.

25.-48. (canceled)
